(19) United States
(12) Patent Application Publication
(10) Pub. No.: US 2025/0319067 A1
(43) Pub. Date: Oct. 16, 2025

(54) Improved Pharmacokinetics of Tryptamine Prodrugs

(71) Applicant: Reunion Neuroscience, Inc., Morristown, NJ (US)

(72) Inventors: Nathan Bryson, Toronto (CA); Beatrix Taylor, Seattle, WA (US); Jasna Hocevar-Trnka, Bellevue, WA (US); Robert Alexander, Paradise Valley, AZ (US)

(21) Appl. No.: 19/251,706

(22) Filed: Jun. 26, 2025

Related U.S. Application Data
(63) Continuation of application No. PCT/CA2024/050007, filed on Jan. 4, 2024.
(60) Provisional application No. 63/478,593, filed on Jan. 5, 2023.
(60) Provisional application No. 63/505,237, filed on May 31, 2023.
(60) Provisional application No. 63/507,600, filed on Jun. 12, 2023.

(51) Int. Cl. A61K 31/4045; A61K 9/00; A61P 25/24

(52) U.S. Cl. CPC A61K 31/4045; A61K 9/0019; A61P 25/24

(57) ABSTRACT

There is described an injectable composition comprising a compound of Formula (I):

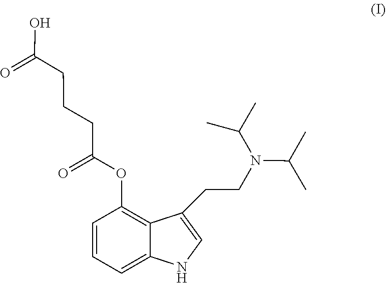 [SMILES: CC(C)N(CCc1c[nH]c2cccc(OC(=O)CCCC(=O)O)c12)C(C)C]

or a pharmaceutically acceptable salt thereof, wherein the compound of Formula (I) is present at a dose of from about 30 mg to about 50 mg calculated as the free base, together with a pharmaceutically acceptable carrier. Also described is a method of treating a mental disorder comprising the step of administering to a patient in need of treatment a pharmaceutical composition comprising the compound of Formula (I) or a pharmaceutically acceptable salt thereof.

### CROSS-REFERENCE TO RELATED APPLICATION

The present application claims the benefit under 35 U.S.C. § 119(e) of provisional patent application Ser. No. 63/478,593, filed Jan. 5, 2023, provisional patent application Ser. No. 63/505,237, filed May 31, 2023 and provisional patent application Ser. No. 63/507,600, filed Jun. 12, 2023, the contents of each of which are hereby incorporated by reference.

### BACKGROUND OF THE INVENTION

### Field of the Invention

In one of its aspects, the present invention relates to a method for treating a mental disorder in patient. In another of its aspects, the present invention relates to an injectable composition, preferably for method for treating a mental disorder in a patient.

### Description of the Prior Art

Tryptamines are a class of 3-aminoethyl-indoles that bind and activate the serotonin receptor, also called the 5HT receptor. A psychedelic state may be achieved by activation of the 2A form of the serotonin receptor by 5HT2A receptor agonist compounds. The endogenous substance for this receptor is 5-hydroxy-tryptamine (serotonin). The tryptamine 3-(2-aminoethyl)-indole is also an endogenous neurotransmitter.

The serotonin receptor system is implicated in depression and depressive states which are commonly treated with 5HT1A antagonists (Affective Disorders: Depression in Neuropsychopharmacology and Therapeutics, Chapter 6, First Edition. Ivor S. Ebenezer, 2015). More recently, 5HT2A agonists have shown potential as medicines for depression (Carhart-Harris 2018 Psychopharmacology).

Tryptamine molecules which produce a psychedelic state and which have been used in traditional medicine, may have therapeutic potential for the treatment of mood disorders, distress, depression and others. For example, ayahuasca is a natural form of dimethyltryptamine (DMT) which when combined with a monoamine oxidase inhibitor can be ingested and produces a variable, but prolonged psychedelic state that can last for 6 to 15 hours. DMT is also naturally found to occur in small amounts in the brain and may act as a neurotransmitter.

Lysergic acid diethylamide (LSD), is a diethylamide derivative of a naturally occurring substance from fungus found in rye grain, which also produces a prolonged psychedelic state up to 8 to 12 hours long.

Psilocybin is a naturally occurring plant-based tryptamine found in Psilocybe mushrooms, and produces a prolonged psychedelic state of about 6 to 8 hours. Psilocybin was first synthesized in 1958 and is currently being investigated as a treatment for depression. Psilocybin is a prodrug, with psilocin being the active species in vivo. Psilocybin contains a phosphate bound to the 4-hydroxy group of psilocin, which is cleaved in the gut when Psilcybe mushrooms or the drug substance is taken orally:

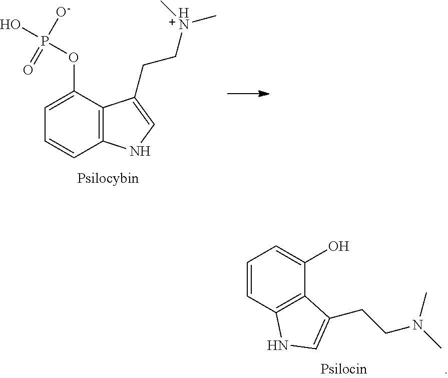

Simple mono-functional organic esters of psilocin have been reported. Lower alkoxy radical modified psilocins have also been described. Sulfate-bound psilocin has been produced and other mono- and di-basic mineral acid modified psilocins have been described. Psilocin acetate is known and has been used in underground psychedelic subculture.

Psychedelic substances have been shown to be effective for treating depression, and even more effective for treating depression when associated with psychotherapy (Watts 2020 Journal of Contextual Behavioral Science).

A limited number of synthetic tryptamine substances have been prepared since perhaps the earliest recorded work of Albert Hoffman. Structure-activity relationships have been described for a variety of tryptamine substances (Claire 1988).

Succinate and other diacid functions have been explored as components of a prodrug delivery system toward water-soluble, injectable forms of hydrophobic or poorly water soluble drug substances, such as testosterone, haloperidol, chloramphenicol or estradiol (Silverman and Holladay, Chapter 9.2: Prodrugs and Drug Delivery Systems in The Organic Chemistry of Drug Design and Drug Action (3^{rd} Ed), 2014). Tetrahydrocannabinol ester of succinic acid has been patented to treat glaucoma. However, ester cleavage is not consistently rapid, is not predictable and can depend on the structure of the moiety attached to the drug and therefore must be investigated (Anderson 1984 JPharmaSci). Esterase enzymes are responsible for active cleavage of the prodrug ester group in vivo and species differences in esterase quantities and specificity in various tissues complicate investigations and optimizations (Bahar 2012 JPharmSci).

4-Glutaroyl-3-(2-diisopropylaminoethyl)-1H-indole hydrochloride—also referred to in this specification as RE104 HCl (formally FT-104)) is a pro-drug of the synthetic psychedelic drug 3-(2-diisopropylamino-ethyl)-1H-indol-4-ol (also called 4-OH-DiPT or isoprocin), a molecule structurally related to psilocin:

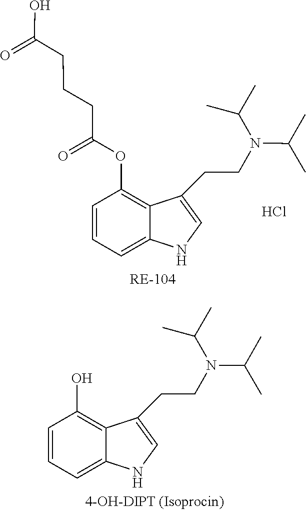 [SMILES: CC(C)N(CCc1c[nH]c2cccc(OC(=O)CCCC(=O)O)c12)C(C)C ; Cl ; CC(C)N(CCc1c[nH]c2cccc(O)c12)C(C)C]

RE104 HCl converts to 4-OH-DiPT in vivo by esterases and is being developed as a SC injection to ensure rapid, complete absorption and to ensure a short and reproducible psychedelic experience with the drug.

The active molecule, 4-OH-DiPT, acts as a serotonin (5-HT) agonist, notably on 5-HT2A and 5-HT2B subtypes, and these receptors have been suggested to contribute to 4-OH-DiPT mediated activity. 4-OH-DiPT belongs to the group of tryptamines, and more specifically, the 4-substituted tryptamines to which the well-known psychedelic compound psilocybin also belongs. Psilocybin is a natural product produced by numerous species of Psilocybe mushrooms. The phosphate group at the 4-position of the psilocybin tryptamine is enzymatically cleaved in the body to produce psilocin, an agonist at a variety of serotonin receptors, the most important of which is the 5-HT2A receptor which is suggested to account for its psychedelic activity (Nichols, 2004). In RE104 HCl, a glutaric acid group at the 4-position of the tryptamine hydrolyzes to form 4-OH-DiPT, which is responsible for the agonist activity at 5-HT2A.

Classic serotonergic hallucinogenic drugs, a group of compounds which bind to 5-hydroxytryptamine (5-HT) receptors, are characterized by their capability to induce changes in sensory perception, emotion, thought, and sense of self, leading to remodeling in mental functions (Vollenweider, 2001; Kometer et al, 2012; Vollenweider et al., 1998) and referred to as mystical-type experiences occurring during psilocybin treatment. These changes have been repeatedly observed to predict subsequent effects on behavior and emotions, including reductions in depressive and anxious behavior (Griffiths et al., 2011; Griffiths et al., 2016; Ross et al., 2016).

Several lines of evidence suggested that serotonergic hallucinogens, such as psilocin and isoprocin (4-OH-DiPT), have clinical potential for inducing therapeutically beneficial behavior changes in a variety of psychiatric conditions. Enduring changes in attitudes, depression, anxiety, wellbeing, substance misuse, and mindfulness have been documented after administration of a psychedelic. Mystical experiences, connectedness, emotional breakthrough and increased neural entropy are related to these long-term changes in psychological functioning (Aday et al., 2020). Additionally, there is emerging evidence that psychedelic-assisted psychotherapy can be a potent treatment for depression and other psychological disorders (Carhart-Harris et al., 2016).

It is also reported that cancer patients often develop chronic, clinically significant symptoms of depression and anxiety which are associated with negative psychiatric and medical outcomes (Swift et al., 2017; Griffiths et al., 2016; Ross et al., 2016).

Promising results have also been obtained in a study in patients with an obsessive-compulsive disorder (OCD) (Moreno et al., 2006) in which subjects were given up to four administrations of psilocybin separated by at least one week, in an escalating dosage sequence of 100 μg/kg, 200 μg/kg, and 300 μg/kg. Decreases in OCD symptoms scores were observed in all subjects at one or more of the testing sessions, and decreases ranged from 23 to 100%.

The efficacy of psilocybin has also been explored in alcohol dependence (Bogenschutz et al., 2015). A recent (2021) review manuscript assessed the clinical effects of serotonergic psychedelics and their therapeutic effect for mental health conditions (Andersen et al., 2021). The literature review included 16 papers that represented 10 independent psychedelic-assisted therapy trials (7 with psilocybin, 2 with ayahuasca and 1 with LSD), and involved 188 patients suffering from cancer or illness-related anxiety and depression disorders including major depressive disorder, OCD or substance use disorder. While the small sample size and the open-nature of the most reviewed studies call for caveat when evaluating efficacy of psychedelic-assisted therapies, comparable efficacies were reported in the two larger controlled studies assessing depressive (and anxiety) symptoms in cancer patients, using non-active placebo or low dose of psilocybin as control (Andersen et al., 2021).

Lastly, anecdotal evidence regarding the effects of 4-OH-DiPT (isoprocin) (10 to 20 mg orally) are provided by Shulgin, who indicates the drug is broadly comparable to other serotonergic psychedelics such as psilocin when used recreationally but is distinguished by its relative brevity of action (Shulgin and Shulgin, 1997). Shulgin (Shulgin and Shulgin, 1997), in referring to oral administration of isoprocin, stated that he “doubt(s) that there is another psychedelic drug, anywhere that can match this one for speed, for intensity, for brevity”. The onset and duration of effect was suggested to be dose related with only a mild physical awareness within 1 hour after 10 mg orally and a quicker onset of effect within 15 minutes at 20 mg. Following the 20 mg oral dose, recovery was indicated to be by 3 h. Controlled studies on the safety, tolerability and pharmacokinetics of 4-OH-DiPT in humans, however, are lacking, in part due to the lack of stability (readily oxidized in air) and very poor solubility (<2 mg/ml in PBS) of the compound.

Conversion to a prodrug RE104 provides improved stability and solubility, while also providing a means of cleavage via esterases to generate the active compound 4-OH-DIPT in situ (in vivo) [see U.S. Ser. No. 11/292,765]. However, what is needed is a clearer understanding of the pharmacokinetics of RE104 or a pharmaceutically acceptable salt thereof (such as the HCl) in humans in relation ot 4-OH-DiPT and psilocybin. It is also desirable to have a clearer understanding of dosing in humans in relation to RE104 or a pharmaceutically acceptable salt thereof (such as the HCl salt).

### SUMMARY OF THE INVENTION

It is an object of the present invention to obviate or mitigate at least one of the above-mentioned disadvantages of the prior art.

It is an object of the present invention to provide improved delivery and pharmacokinetics following administration of RE104 or a pharmaceutically acceptable salt thereof (such as the HCl salt).

It in another object of the current invention to provide a shorter duration of psychedelic experience following administration of the pro-drug of RE104 or a pharmaceutically acceptable salt thereof (such as the HCl salt) at levels that produce a complete mystical experience, defined as ≥60% for each domain of the Mystical Experience Questionnaire (MEQ-30), in a majority of subjects. [see in Ko, K et al. Front. Psychiatry 2022, 13, 917199].

It in another object of the current invention to provide a shorter duration of psychedelic experience following administration of the pro-drug of RE104 or a pharmaceutically acceptable salt thereof (such as the HCl salt) at levels expected to produce therapeutic effect as compared to administration of psilocybin at effective dose levels.

It is still another object of the current invention to provide fewer side effects, in particular blood pressure and headache following administration of the pro-drug of RE104 or a pharmaceutically acceptable salt thereof (such as the HCl) at levels expected to produce therapeutic effect as compared to administration of psilocybin at effective dose levels.

It is yet another object of the current invention to provide improved safety and tolerability following administration of the pro-drug of RE104 or a pharmaceutically acceptable salt thereof (such as the HCl) at levels expected to produce therapeutic effect as compared to administration of psilocybin at effective dose levels.

In one of its aspects, the invention relates to a composition comprising a compound described herein, and a pharmaceutically acceptable excipient. In some embodiments, the composition comprises an oral dosage formulation or an injectable formulation.

In another aspect, the invention comprises a method of treating a mental disorder, comprising the step of administering an effective amount of a compound described herein. In some embodiments, the mental disorder is a depressive condition, including unipolar and bipolar depressive conditions, such as but not limited to depression, depression from generalized anxiety, major depression, treatment resistant depression and postpartum depression.

In another aspect, the invention relates to the use of a compound described herein to treat a mental disorder, or in the manufacture of a medicament for treating a mental disorder, such as depression.

In another aspect, the invention relates to a method of treating a mental disorder comprising the step of administering to a patient in need of treatment a pharmaceutical composition comprising a compound of Formula (I):

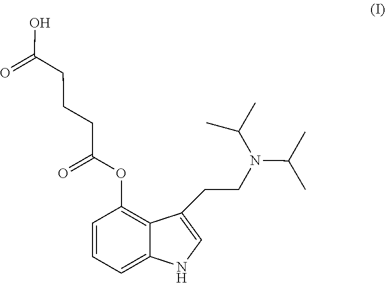 [SMILES: CC(C)N(CCc1c[nH]c2cccc(OC(=O)CCCC(=O)O)c12)C(C)C]

or a pharmaceutically acceptable salt there of, wherein the compound of Formula (I) is present at a dose of from about 30 mg to about 50 mg calculated as the free base, together with a pharmaceutically acceptable carrier.

In another aspect, the invention relates to a method of treating a mental disorder comprising the step of administering to a patient in need of treatment a pharmaceutical composition comprising a compound of Formula (I):

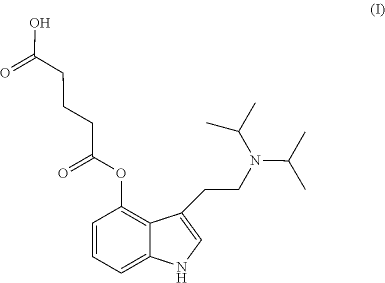 [SMILES: CC(C)N(CCc1c[nH]c2cccc(OC(=O)CCCC(=O)O)c12)C(C)C]

or a pharmaceutically acceptable salt there of, wherein the compound of Formula (I) is present at a dose of 30 mg or about 40 mg calculated as the free base, together with a pharmaceutically acceptable carrier.

In another aspect, the invention relates to an injectable composition comprising a compound of Formula (I):

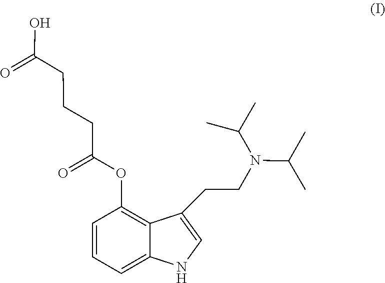 [SMILES: CC(C)N(CCc1c[nH]c2cccc(OC(=O)CCCC(=O)O)c12)C(C)C]

or a pharmaceutically acceptable salt there of, wherein the compound of Formula (I) is present at a dose of 1 mg/kg calculated as the free base, together with a pharmaceutically acceptable carrier.

In another aspect, the invention relates to an injectable composition comprising a compound of Formula (I):

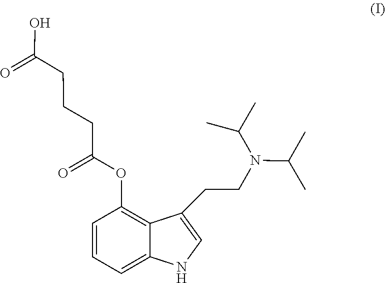 [SMILES: CC(C)N(CCc1c[nH]c2cccc(OC(=O)CCCC(=O)O)c12)C(C)C]

or a pharmaceutically acceptable salt there of, wherein the compound of Formula (I) is present at a dose of from about 30 mg to about 50 mg calculated as the free base, together with a pharmaceutically acceptable carrier.

In another aspect, the invention relates to an injectable composition comprising a compound of Formula (I):

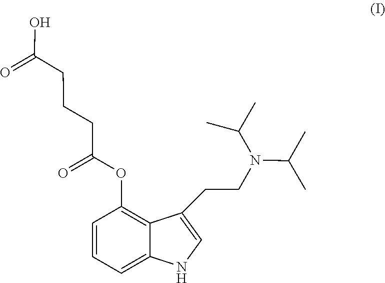 [SMILES: CC(C)N(CCc1c[nH]c2cccc(OC(=O)CCCC(=O)O)c12)C(C)C]

or a pharmaceutically acceptable salt there of, wherein the compound of Formula (I) is present at a dose of about 30 mg or about 40 mg calculated as the free base, together with a pharmaceutically acceptable carrier.

The administration of the pro-drug of RE104 or a pharmaceutically acceptable salt thereof (such as the HCl) described herein may be useful to treat mental disorders, such as a depressive condition, including unipolar and bipolar depressive conditions, such as but not limited to depression, depression from generalized anxiety, major depression, treatment resistant depression and postpartum depression.

### BRIEF DESCRIPTION OF THE DRAWINGS

Embodiments of the present invention will be described with reference to the accompanying drawings, in which:

FIG. 1 illustrates a schematic of the design of the human clinic study described in Example 2;

FIG. 2 illustrates the Modified Drug Effects Questionaire (DEQ) used in the human clinic study described in Example 2;

FIG. 3 illustrates the Revised Mystical Experience Questionare (MEQ) used in the human clinic study described in Example 2;

FIG. 4, illustrates the mean DEQ-high scores of over time by dose in the human clinic study described in Example 2;

FIG. 5 illustrates the mean MEQ score by individual domain and total score per RE104 dose level in the human clinic study described in Example 2;

FIG. 6 illustrates the mean plasma 4-OH-DiPT concentrations and DEQ-high scores over time for the RE104 33-mg dose group in the human clinic study described in Example 2;

FIG. 7 illustrates the mean plasma 4-OH-DiPT concentrations versus DEQ-high scores for all Cohorts in the human clinic study described in Example 2;

FIG. 8 illustrates 5-HT2A binding for RE104 in the study described in Example 3;

FIG. 9 illustrates 5-HT2A binding for 4-OH-DiPT in the study described in Example 3;

FIG. 10 illustrates 5-HT2B binding for RE104 in the study described in Example 3;

FIG. 11 illustrates 5-HT2A activation for RE104 in the study described in Example 3;

FIG. 12 illustrates mouse plasma concentration of RE104 over time in the study described in Example 3;

FIG. 13 illustrates rat plasma concentration of RE104 over time in the study described in Example 3;

FIG. 14 illustrates dog plasma concentration of RE104 over time in the study described in Example 3;

FIG. 15 illustrates human plasma concentration of RE104 over time in the study described in Example 3;

FIG. 16 illustrates mean 4-OH-DiPT plasma concentration and mean number head twitch responses (HTRs) counted over 10-minute intervals as a function of time after intravenous (IV) and subcutaneous (SC) administration of RE104 or 4-OH-DiPT to groups of 3 rats in the study described in Example 3;

FIG. 17 illustrates the correlation between HTRs and mean 4-OH-DiPT plasma concentration for 2 mg/kg RE104 intravenous administration (line represents the curve of best fit) in the study described in Example 3;

FIG. 18 illustrates the correlation between HTRs and mean 4-OH-DiPT plasma concentration for 2 mg/kg RE104 subcutaneous administration (line represents the curve of best fit) in the study described in Example 3;

FIG. 19 illustrates the correlation between HTRs and mean 4-OH-DiPT plasma concentration for 1.39 mg/kg 4-OH-DiPT intravenous administration (line represents the curve of best fit) in the study described in Example 3;

FIG. 20 illustrates the correlation between HTRs and mean 4-OH-DiPT plasma concentration for 1.39 mg/kg 4-OH-DiPT subcutaneous administration (line represents the curve of best fit) in the study described in Example 3; and

FIGS. 21-23 each illustrate the effect of RE104 and RE109 (psilocin glutarate) on specific behavior (immobility time in FIG. 21, swimming time in FIG. 22 and climbing time in FIG. 23) in the forced swim test (n=12 male Wistar rats per treatment group) [error bars represent the standard error of the mean. *P<0.05, **P<0.01, ***P<0.001 relative to vehicle by 1-way analysis of variance followed by Dunnett's multiple comparison test. ††P<0.01 relative to vehicle by unpaired t test. ‡P<0.05, ‡‡‡P<0.001 relative to RE109].

### DETAILED DESCRIPTION OF THE PREFERRED EMBODIMENTS

In one of its aspects, the present invention relates to a method of treating a mental disorder comprising the step of administering to a patient in need of treatment a pharmaceutical composition comprising a compound of Formula (I):

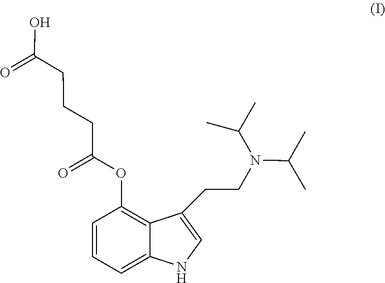 [SMILES: CC(C)N(CCc1c[nH]c2cccc(OC(=O)CCCC(=O)O)c12)C(C)C]

or a pharmaceutically acceptable salt there of, wherein the compound of Formula (I) is present at a dose of from about 30 mg to about 50 mg calculated as the free base (e.g., about 30 mg or about 40 mg calculated as the free base), together with a pharmaceutically acceptable carrierat a dose of from about 30 mg to about 50 mg, together with a pharmaceutically acceptable carrier.

Preferred embodiments of this method may include any one or a combination of any two or more of any of the following features:
the compound of Formula (I) is used in the form of the hydrochloride salt thereof;
when used as the hydrochloride salt, the compound of Formula (I) is used at a dose of about 33 mg or about 44 mg;
the compound of Formula (I) has a duration of action of from about 2.5 hours to about 4.5 hours after administration;
the compound of Formula (I) has a duration of action of about 3.5 hours or 3.7 hours after administration;
the compound of Formula (I) has a Cₘₐₓ in the patient in the range of from about 1000 ng/mL to about 2000 ng/mL;
the compound of Formula (I) has a Cₘₐₓ in the patient in the range of from about 1500 ng/mL to about 1800 ng/mL;
the compound of Formula (I) has a Tₘₐₓ in the patient in the range of from about 20 minutes 30 minutes;
the compound of Formula (I) has a Tₘₐₓ in the patient in the range of about 15 minutes;
the compound of Formula (I) is converted to a compound of Formula (II) after administration

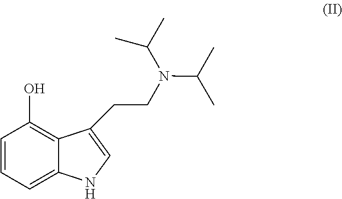 [SMILES: CC(C)N(CCc1c[nH]c2cccc(O)c12)C(C)C]

and the compound of Formula (II) has a Cₘₐₓ in the patient in the range of from about 100 ng/mL to about 300 ng/mL;
the compound of Formula (I) is converted to a compound of Formula (II) after administration

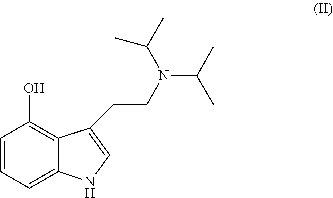 [SMILES: CC(C)N(CCc1c[nH]c2cccc(O)c12)C(C)C]

and the compound of Formula (II) has a Cₘₐₓ in the patient in the range of from about 120 ng/mL to about 300 ng/mL;
the compound of Formula (I) is converted to a compound of Formula (II) after administration

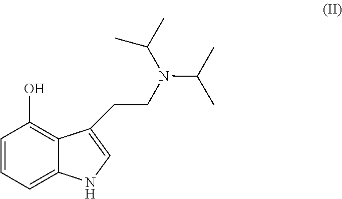 [SMILES: CC(C)N(CCc1c[nH]c2cccc(O)c12)C(C)C]

and the compound of Formula (II) has a Tₘₐₓ in the patient in the range of from about 60 minutes to about 150 min.
the compound of Formula (I) is converted to a compound of Formula (II) after administration

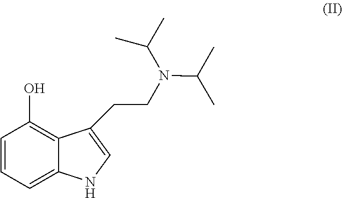 [SMILES: CC(C)N(CCc1c[nH]c2cccc(O)c12)C(C)C]

and the compound of Formula (II) has a Tₘₐₓ in the patient in the range of from about 60 minutes to about 80 min;
the method demonstrates an average MEQ30 score in the patient of at least 50%; and/or
the method demonstrates an average MEQ30 score in the patient of at least 60%;
the pharmaceutical composition is administered to a patient by injection;
and/or
the pharmaceutical composition is administered to a patient by subcutaneous injection.

As used herein, the term “mental disorder” includes those disorders which may be diagnosed by a mental health professional as a psychological or psychiatric disorder, including those which may be diagnosed by reference to Diagnostic and Statistical Manual of Mental Disorders (DSM-5).

The term “treating”, “treat” or “treatment” as used herein embraces both preventative, i.e., prophylactic, and palliative treatment, i.e., relieve, alleviate, or slow the progression of the patient's disease, disorder or condition.

As used herein, “psychedelic state” is an altered state of consciousness experienced by a person, which may include intensified sensory perception, perceptual distortion or hallucinations, and/or feelings of euphoria or despair. Psychedelic states have been described as resulting from psychedelic drugs such as DMT (dimethyltryptamine), LSD, mescaline or psilocybin. Other known psychedelic drugs include the 4-hydroxy analogs of N-Methyl-N-isopropyltryptamine (MiPT) and N,N-diisopropyltryptamine (DiPT).

The present invention comprises the use of prodrugs of hydroxyindole 5HT2A agonists such as RE104 which induce a psychedelic state or which still provide a beneficial therapeutic effect without being associated with a psychedelic state. The prodrugs may be used in combination with other treatments known to be effective for treating mental disorders, such as psychotherapy, electroconvulsive therapy and/or other pharmaceutical compounds, for example, with concomitant use of tricyclic antidepressants (TCAs), selective serotonin reuptake inhibitors (SSRIs), selective norepinephrine reuptake inhibitors (SNRIs), monoamine oxidase inhibitors (MOAIs) or other anti-depressants. In preferred embodiments, the treatment may produce lasting effects, for example longer than 1 month after a single treatment, preferably longer than 3 months, and more preferably longer than 6 months. In some embodiments, additional therapy may not be required.

### Compounds

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. The expression “prodrug” refers to compounds that are drug precursors which following administration, release the drug (or “active”) in vivo via some chemical or physiological process (e.g., hydrolysis, enzymatic cleavage or hydrolysis, or metabolism is converted to the desired drug form). The invention includes within its scope the pharmaceutically acceptable salts of the compounds of the invention. Accordingly, the phrase “or a pharmaceutically acceptable salt thereof” is implicit in the description of all compounds described herein unless explicitly indicated to the contrary.

In some embodiments, the compounds used in the composition and method of treatment of the present invention comprise prodrug compounds that are readily purified, formulated and stable, and preferably may be used to provide highly soluble drug substances, with fast onset and elimination for convenient use in a clinical setting. In some embodiments, the compounds may be produced as a zwitterion, which may be converted to a pharmaceutically acceptable salt.

In some embodiments, the compounds of the present invention preferably allow for fast cleavage in vivo of the prodrug moiety to give the active pharmacophore, for example, 90% conversion may occur in under 4 hours, preferably in less than 2 hours, and more preferably in less than 1 hour. Prodrugs may have lesser, little or no pharmacological activity themselves, however when administered to a patient, may be converted into an active compound, for example, by hydrolytic cleavage.

### Formulations and Compositions

The invention also provides pharmaceutically acceptable compositions which comprise a therapeutically effective amount of one or more of the compounds described herein, formulated together with one or more pharmaceutically acceptable carriers (additives) and/or diluents, and optionally, one or more additional therapeutic agents. While it is possible for a compound described herein to be administered alone, it is preferable to administer the compound as a pharmaceutical composition.

The term “pharmaceutical composition” means a composition comprising a compound of the invention in combination with at least one additional pharmaceutically acceptable carrier. A “pharmaceutically acceptable carrier” refers to media generally accepted in the art for the delivery of biologically active agents to animals, in particular, mammals, including, i.e., adjuvant, excipient or vehicle, such as diluents, osmotic complement, preserving agents, fillers, flow regulating agents, disintegrating agents, wetting agents, emulsifying agents, suspending agents, sweetening agents, flavoring agents, perfuming agents, antibacterial agents, antifungal agents, lubricating agents, polymers, solubilizing agents, stabilizers, antioxidants and dispensing agents, depending on the nature of the mode of administration and dosage forms. Each carrier must be “acceptable” in the sense of being compatible with the other ingredients of the formulation and not injurious to the patient.

As used herein, “oral” administration includes swallowing for ingestion in the stomach or gut, and further includes lingual, sublingual, buccal and oropharyngeal administration. The compounds of this invention can be administered for any of the uses or methods described herein by any suitable means, for example, orally, such as tablets, capsules (each of which may include sustained release or timed release formulations), pills, powders, granules, elixirs, suspensions (including nano suspensions, micro suspensions, spray-dried dispersions), syrups, and emulsions; sublingually (e.g. as thin films, effervescent tablets or tablets that dissolve spontaneously under the tongue); parenterally, such as by subcutaneous, intravenous, intramuscular injection, or infusion techniques (e.g., as sterile injectable aqueous or non-aqueous solutions or suspensions); nasally, including administration to the nasal membranes, such as by inhalation spray; or rectally such as in the form of suppositories.

A preferred formulation allows for subcutaneous injection. Likewise, preferred injection pathways are subcutaneous.

The dosage regimen for the compounds described herein will, of course, vary depending upon known factors, such as the pharmacokinetic and pharmacodynamic characteristics of the particular agent and its mode and route of administration; the species, age, sex, health, medical condition and weight of the recipient; the nature and extent of the symptoms; the kind of concurrent treatment; the frequency of treatment; the route of administration, the renal and hepatic function of the patient; and, the effect desired. The selected dosage level may also depend on the additional factors including the activity of the particular compounds and pharmaceutical compositions described herein, whether an ester, salt or amide substituent is of the compound is used, the time of administration, the rate of excretion or metabolism of the particular compound being employed, the rate and extent of absorption, the duration of the treatment, other drugs that may be administered to the patient, compounds and/or materials used in combination with the particular compound employed and like factors well known in the medical arts.

Generally, the dosage of the prodrug for a therapy session, when used for the indicated effects, will range between about 0.001 to about 500 mg per dose, preferably between about 0.01 to about 200 mg per dose, and preferably between about 0.1 to about 50 mg per dose, such as 10, 20, 30, 40, 50, 100 or 200 mg. In more preferred embodiments, the dosage of the prodrug for a therapy session, when used for the indicated effects, about 30 mg to about 50 mg per dose, such as 33 mg or 44 mg.

Intravenously, the most preferred doses will range from about 0.01 to about 10 mg/kg/minute during a constant rate infusion.

Compounds of this invention may be administered in a single daily dose, or the total daily dosage may be administered in multiple divided doses, such as two, three, or four times daily. Alternatively, the doses may be provided on a weekly, biweekly, or monthly basis. In a preferred embodiment, only one or two doses are required for an anti-depressant effect than may extend for 1, 2, 3 or 6 months, or more.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include antioxidants, colorants, flavoring agents, preservatives and taste masking agents.

Exemplary tablets contain up to about 80 wt % drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet, dry, or melt granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0 8247 6918 X).

A typical capsule for oral administration contains at least one of the compounds of the present invention (e.g. 25 mg), lactose (e.g. 75 mg), and magnesium stearate (e.g. 15 mg). The mixture is passed through a 60 mesh sieve and packed into a No. 1 gelatin capsule.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds used in the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle free injectors and infusion techniques.

Parenteral formulations are typically aqueous solutions which may contain excipients such as salts, carbohydrates and pH adjusting or buffering agents (preferably to a pH of from 3.0 and 7.0, preferably 4.0 to 6.0, and more preferably 4.5 to 5.5), but, for some applications, they may be more suitably formulated as a sterile non aqueous solution or as a dried form to be used in conjunction with a suitable vehicle such as sterile, pyrogen free water or pre-fabricated, ready-to-mix aqueous buffer. Osmotic agents may be included to control tonicity.

The preparation of parenteral kits for reconstitution at point-of-care under sterile conditions, for example, by lyophilization, may readily be accomplished using standard pharmaceutical techniques well known to those skilled in the art.

A typical injectable preparation is produced by aseptically placing at least one of the compounds of the present invention (e.g., 25 mg, 33 mg or 44 mg) into a vial as a sterile filtered solution, aseptically freeze-drying and sealing. For use, the contents of the vial are mixed with, for example, 2 mL of physiological saline for injection, optionally with an appropriate amount of osmotic complements and pH adjusters to achieve a slightly acidic to neutral pH (e.g. pH 4-7), to produce an injectable preparation with low irritation but retain solubility and/or stability of the prodrug.

Compounds used in the invention may be combined with soluble macromolecular entities, such as cyclodextrin and suitable derivatives thereof or polyethylene glycol containing polymers, in order to improve their solubility, dissolution rate, taste masking, bioavailability and/or stability for use in any of the aforementioned modes of administration.

Drug cyclodextrin complexes, for example, are found to be generally useful for most dosage forms and administration routes. Both inclusion and non inclusion complexes may be used. As an alternative to direct complexation with the drug, the cyclodextrin may be used as an auxiliary additive, i.e. as a carrier, diluent, or solubilizer. Most commonly used for these purposes are alpha, beta and gamma cyclodextrins, examples of which may be found in International Publication Nos. WO 91/11172, WO 94/02518 and WO 98/55148.

Regardless of the route of administration selected, the compounds used the present invention, which may be used in a suitable hydrated form, and/or the pharmaceutical compositions of the present invention, are formulated into pharmaceutically acceptable dosage forms by conventional methods known to those of skill in the art. Actual dosage levels of the active ingredients in the pharmaceutical compositions of this invention may be varied so as to obtain an amount of the active ingredient which is effective to achieve the desired therapeutic response for a particular patient, composition, and mode of administration.

A person (e.g,. a nurse, a nurse practitioner, a physician or veterinarian) having ordinary skill in the art can readily determine and prescribe the effective amount of the pharmaceutical composition required. For example, such a person could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

As used herein, a “therapeutically effective amount” refers to that amount of a compound being administered which will relieve to some extent one or more of the symptoms of the disorder being treated. In reference to the treatment of depression, a therapeutically effective amount refers to that amount which has the effect of reducing the severity of depression. Depression severity may be assessed using well-known structured assessment tools such as Structured Clinical Interview for DSM-5 (SCID-5) and the GRID-Hamilton Depression Rating Scale (GRID-HAMD). A therapeutically effective amount may be less than that required for a psychedelic state.

An effective dosage can be administered in one or more administrations. For the purposes of this invention, an effective dosage of drug, compound, or pharmaceutical composition is an amount sufficient to accomplish prophylactic or therapeutic treatment either directly or indirectly. As is understood in the clinical context, an effective dosage of drug, compound or pharmaceutical composition may or may not be achieved in conjunction with another therapy, drug, compound or pharmaceutical composition.

### Therapeutic Methods and Uses

Treatment with the compounds of the invention, such as RE104 described herein may substantially alleviate clinical or subclinical depression and may avoid relapse, particularly if used in combination with psychotherapy for the treatment of depression. It is known that administration of an effective dose of psilocybin produced rapid and large reductions in depressive symptoms, and many subjects achieve remission through a four-week follow up (Davis et. al.) Without restriction to a theory, it is believed that the psychedelic state is associated with the beneficial effects, however, some compounds which are 5HT2A agonists may provide the desired therapeutic effect without the psychedelic state. One aspect of the invention comprises prodrugs of those 5HT2A agonists which do provide a beneficial therapeutic state.

In general, the present invention includes the use of a compound of the present invention herein, to treat any disease or disorder which may be alleviated by a 5HT2A agonist, or the use of a compound of the present invention herein to manufacture a medicament to treat any disease or disorder which may be alleviated by a 5HT2A agonist, or a method of treating any disease or disorder which may be alleviated by a 5HT2A agonist.

In some embodiments, the invention may comprise the use of the compounds described herewin to treat mental disorders. In some embodiments, the invention may comprise the use of the compounds of the present invention to treat depression, and particularly major depressive disorder, postpartum depression and treatment resistant depression. Other conditions that may be treated include: anxiety disorders, including anxiety in advanced stage illness (e.g., cancer) as well as generalized anxiety disorder, cluster headaches, obsessive compulsive disorder, personality disorders including conduct disorder, drug disorders including alcohol dependence, nicotine dependence, opioid dependence, cocaine dependence and other addictions including gambling disorder, eating disorder and body dysmorphic disorder, chronic pain, or chronic fatigue.

In some embodiments, the invention may comprise a method of treating mental disorders comprising administering to a subject in need thereof a therapeutically effective amount of a compound of the present invention. In one embodiment, there is provided a method of treating depression comprising administering to a subject in need thereof therapeutically effective amount of a compound of the present invention. The depression may be drug-resistant depression or major depressive disorder.

For example, a patient diagnosed with depression may be screened prior to treatment, and then prepared for a dosing session by a trained psychotherapist. Within a dosing session, a compound of the present invention may be administered by injection of a sterile solution at a rate of 0.01-0.3 mg/kg to the patient. The patient is preferably seated for the duration of the session while being blindfolded. For safety, a trained health care professional may monitor the patient throughout the dosing session, which may last up to 12 hours. In some cases, music may be played for the patient. When the health care professional can determine that the drug substance has cleared, the trained health care professional may assist the patient with any questions relating to the psychedelic experience, and then the patient may be discharged.

To further alleviate any anxiety that may occur relative to therapy, the physician may prefer to divide the therapeutic dose and thereby reduce the initial onset of psychoactivity before applying the full complement of the dosage to achieve the full effect.

In some embodiments, treatment with a compound of the present invention may be combined with concomitant treatment with another anti-depressant drugs, either concurrently or consecutively. In preferred embodiments, treatment with a compound of the present invention is combined with psychotherapy, which may be applied prior to or after treatment. If prior to, the session may focus the patient on the intent of treatment. If after, psychotherapy is preferably performed within 48 hours of the dosing session to help the patient integrate any feelings, emotions, visions or thoughts that may have occurred during the session, as well as to allow the psychotherapist may offer advice on how best to change thinking or behavior patterns so as to improve anti-depression outcomes. Psychotherapy may continue as needed after the dosing session, for example, up to an additional 3 months, to help the patient integrate any experiences or learnings that occurred to the patient during the dosing session.

The present invention may be described with reference to the following Examples. These Examples are provided for the purpose of illustration only. All terms, names, abbreviations or acronyms are those commonly understood by those skilled in the art. Compounds shown in their zwitterionic form may readily be visualized in their neutral form by one skilled in the art, and vice versa. The Examples should not be used to construe or limit the scope of the invention.

### Example 1

### Initial Studies and Non-Clinical Pharmacology

RE104 hydrochloride (RE104 HCl) is a pro-drug of the synthetic psychedelic drug 3-(2-diisopropylaminoethyl)-1H-indol-4-ol (4-OH-DiPT or isoprocin), a molecule structurally related to psilocin. 4-OH-DiPT has been reported as an abused substance that produces subjective experiences in a recreational setting that are similar to psilocybin, but with psychoactive properties of shorter duration (Shulgin and Shulgin, 1997). No formal human studies with 4-OH-DiPT have been recorded, but the literature does present limited pharmacological and non-clinical studies. 4-OH-DiPT acts as a serotonin (5-hydroxytryptamine [5-HT]) agonist, notably on 2A and 2B subtypes. Several lines of evidence suggest that serotonergic hallucinogens, such as those following administration of psilocybin (of which the active is psilocin) and isoprocin, have clinical potential for inducing therapeutically beneficial behavioral changes in a variety of psychiatric conditions.

For all studies, placebo was a commercially available 0.9% saline solution. All drug administration for the following studies is a single subcutaneous (SC) injection, for exampled in the upper arm of the patient. Without being limitive as to the route of administration, subcutaneous injection can often be preferred as it provides certain advantages over oral administration, for example, such as (1) ensures dose absorption whereas oral absorption is often associated with nausea and dose can be lost due to vomiting, (2) ensures complete and reproducible absorption where GI absorption can vary with several parameters including metabolic profile of the individual and recent food intake, and (3) ensures fastest and most complete systemic availability by avoiding delays related to GI transit, and (4) these combined factors lead to greater reproducibility in the rate of absorption and overall PK profiles which also ensures as much consistency in duration and intensity of the effects of the drug from one treatment to the next.

### Non-Clinical Pharmacology

Similar to psilocybin and psilocin, RE104 and 4-OH-DiPT are 5-HT2A agonists. Receptor binding studies (5-HT2A and 5-HT2B agonist radioligand assays) have indicated that the active moiety 4-OH-DiPT is more potent (IC50 150 nM and 42 nM at 5-HT2A and 5-HT2B, respectively) than the pro-drug RE104 (IC50 1600 nM and 740 nM at 5-HT2A and 5-HT2B, respectively). Additionally, in vitro radioligand binding assays, RE104 may interact with other receptors (e.g. 5-HT transporter, and kappa and mu opioid receptors) as may 4-OH-DiPT (5HT transporter, kappa opioid, 5-HT1A, 5-HT1B, mu opioid and muscarinic M2 receptors). However engagement at the 5-HT2A and 5-HT2B receptors is dominant for both RE-104 and 4-OH-DiPT compared to all other receptors included in the screening assay.

When administered to rats, 5-HT2A agonists such as psilocybin, psilocin, RE104 and 4-OH-DiPT induce behaviors such as head twitch, wet dog shakes, tremor, hind limb abduction, and backward walking due to exaggerated pharmacology at serotonin receptors (Halberstadt et al., 2011; Haberzettl et al., 2013; Rickli 2016 Eur Neuropharmacol; Klien 2020 ACS Pharmacol Transl Sci). Head twitch in rodents has been correlated to the hallucinogenic response similar to a psychedelic effect in humans (Willins and Meltzer, 1997). It is reported that the exaggerated pharmacology related to acute or chronic overstimulation of the serotonin receptor produces a condition known as serotonin syndrome (SS). Serotonin syndrome is an adverse drug reaction which is the result of excessive serotonergic activity, observed at very high dose levels, such as those evaluated in non-clinical toxicology studies described within the Investigator's Brochure and can occur in humans upon excessive acute or chronic exposure (Boyer and Shannon, 2005). Acute SS associated with serotonin agonists, such as psilocybin, crudely evolve with plasma levels of active drug and resolve within a short period of time (<24 h). Chronic SS can have more consequential health impacts and should be avoided. RE104 is being developed as a single use medication, where repeat, intermittent use will be limited and so only mild to modest actions of exaggerated pharmacology could be expected. See for example Franciscangeli 2019 Int J Mol Sci.

### Safety Pharmacology

In a behavioral study in rats, single SC administration of 10, 25 or 50 mg/kg RE104 HCl induced significant acute, and reversible neuro-pharmacological effects; however, all observations were attributed to exaggerated pharmacologic effects at the serotonin receptor of the test article (i.e., SS). The incidence and duration of observations were dose related. At 10 mg/kg, the effects were observed at 30 minutes (min) to 1 hour (h); at 25 mg/kg, the effects were observed to 6 h, and at 50 mg/kg, effects were noted through 6 h post-dose but were resolved by 24 h.

In a respiratory function study in male rats, at single SC doses of 10, 25, and 50 mg/kg, RE104 HCl produced increases in respiration rate (8-28%) and decreases in tidal volume (28-30%) and minute volume (24-31%). All these changes were transient and returned to pre-dose values by the 6-hour post-dose reading. Based upon the small magnitude and the transient nature of these changes, the changes were not considered adverse.

In a cardiovascular study, RE104 HCl given as a single SC dose of 1 mg/kg caused a small transient increase in heart rate but had no effect on blood pressure in conscious telemetered beagle dogs. Transient increases in heart rate and blood pressure (systolic, diastolic and mean) were observed at 3 and 8 mg/kg doses. No significant biologically relevant effects on cardiac rhythm, electrocardiogram (ECG) parameters or body temperature were observed at SC doses of 1, 3 or 8 mg/kg RE104 HCl.

At the highest concentration tested (30 μM as zwitterion), RE104 HCl exhibited minimal effects in the human ether-a-go-go-related gene (hERG) tail current assay, with a 5.9+1.6% decrease in hERG current amplitude.

### Toxicology

Non-GLP single dose, dose range studies with RE104 HCl were performed in rats and dogs to explore toxicity and establish a maximum tolerated dose (MTD) by SC injection. The rat study explored dosing with two formulations, water for injection (WFI) at pH 3-4 and phosphate buffered saline (PBS) with a final formulation pH of 4.5-5. Single doses of 20 to 300 mg/kg formulated in WFI produced dose-dependent pharmacologically related systemic serotonergic clinical signs. Similarly, SC administration with doses of 25 to 225 mg/kg RE104 formulated in PBS caused clinical signs of SS around 1 hour with recovery by 24 hours. Decreased food consumption, body weight and body temperature (females) were noted at 225 mg/kg.

Injection site reactions were noted at all doses formulated in WFI (20 to 150 mg/kg) and from 75 mg/kg formulated in PBS. Clinical signs were reversible by Recovery Day 13 (Study Day 14). Microscopic injection site pathology was seen following a single dose of 225 mg/kg. The MTD in the rat for RE104 in WFI was 150 mg/kg and in PBS was 75 mg/kg.

In a GLP 14-day extended single SC dose toxicity study in rats, doses of 0 (PBS), 10, 25 and 75 mg/kg were evaluated. Animals were necropsied on Study Day 2 and Day 14 (also referred to as Recovery Day 13) post-dosing.

There were no substantial gender differences (>2-fold) in the systemic exposure (Cₘₐₓ and AUC₀₋ₜ) of RE104 HCl and 4-OH-DiPT. Generally greater than dose-proportional increases in systemic exposure to RE104 and 4-OH-DiPT was seen with increasing dose across the range 10 to 75 mg/kg, except for 4-OH-DiPT Cmax in males, which exhibited less than dose-proportional increases.

Clinical signs of acute, mild SS (“exaggerated pharmacology of the serotonin 2A receptor) were evident in all RE104 HCl dose groups along with reversible changes at the injection site including edema/inflammatory infiltrate in all treated groups at 25 and 75 mg/kg RE104.

In male rats, changes were resolved in all groups on Day 2 and animals were normal.

### Pre-Clinical Pharmacokinetics and Metabolism

The pharmacokinetics (PK) of RE104 and 4-OH-DiPT were characterized after a single intravenous (IV) or SC doses of RE104 HCl at 2 mg/kg in rats. RE104 was only measurable in plasma at 5 min until 0.25 h post-dose following IV administration and was only measurable at 5 min post-dosing following SC dosing; therefore, PK parameters could not be determined for RE104 in this study. The active, 4-OH-DiPT, exhibited a short elimination half-life (t1/2) in plasma for all dose routes, with a t1/2 of 0.426 h (IV) and 0.678 h (SC). The absolute bioavailability of RE104 following SC dosing was close to 100% in rat. In monkeys, RE104 is also converted rapidly to 4-OH-DiPT, and after IV and SC dosing, the half-lives of 4-OH-DiPT were 0.631 h and 0.950 h, respectively. In dog, the conversion of RE104 to 4-OH-DiPT is slower and the half-life of the parent drug RE104 after SC dosing was 1.3 h, while the half-life of 4-OH-DiPT after SC dosing was 0.888 hr. The half-life of 4-OH-DiPT after IV dosing was 0.863 h. In dog, the absolute bioavailability of RE104 upon SC administration is also close to 100%.

The active species from RE104 HCl metabolism, 4-OH-DiPT, has a low plasma protein binding to animal plasma proteins (˜30-60% binding) and showed higher binding to human plasma proteins which was concentration dependent (˜80-93% bound across the range of 1 to 20 PM).

In hepatocytes, the in vitro intrinsic clearance (CLint) values for RE104 were fastest in the rat and slowest in the dog, with CLint in rat, rabbit, human, monkey, and dog of 6.66, 4.88, 3.45, 1.14, and 0.87 mL/hr/106 cells, respectively. In human, rat, rabbit, dog and monkey hepatocytes, the metabolite profile of RE104 was qualitatively similar across these species with only minor differences. The human hepatocyte metabolic profile of 4-OH-DiPT was also qualitatively similar to that of RE104 in rat, rabbit, dog, and monkey hepatocytes with only minor differences. In an in vitro assay using recombinant human enzymes (Supersomes), which included multiple cytochrome P450 (CYP) enzymes, Flavin-containing monooxygenase (FMO), and monoamine oxidase (MAO) enzymes, indicated that RE104 was not a substrate for any isoform of cytochrome P450, FMO or MAO isoforms. The enzymatic conversion of RE104 to 4-OH-DiPT is likely by plasma esterases, such as cholinesterases.

### Dosing Rationale and Exposure Limits

The no observed adverse effect level (NOAEL) for the local injection site changes were judged to be 10 mg/kg in males and female rats. For systemic effects, the NOAEL was 10 mg/kg. The dose of 10 mg/kg in males was considered a low adverse effect level (LOAEL). The 10 mg/kg dose in rat provides a human equivalent dose [HED] of approximately 97 mg (expressed as zwitterion), assuming a 60 kg human body weight and standard (FDA guidance, 2005) body surface area (BSA) conversion factors. This HED dose is approximately 19-fold higher than the proposed starting dose of 5 mg (expressed as zwitterion) and 2-fold higher than the maximum proposed dose of 48 mg (expressed as zwitterion) in this study. At 10 mg/kg, the maximum plasma concentration (Cmax) for RE104 was 0.751 ng/mL, and the area under the concentration-time curve from time zero to the last time point (AUC0-t) was undetermined due to its rapid conversion to 4-OH-DiPT; for 4-OH-DiPT, the mean Cₘₐₓ was 542 ng/mL and the AUC₀₋ₜ was 1230 h·ng/mL. A non-GLP dose range study was performed in the dog in order to determine an MTD. Single SC doses of 7.5, 15 and 20 mg/kg were evaluated. Clinical signs of SS were evident across the dose range with hyper salivation, behavioral and posture changes at all doses.

A GLP 14-day extended single SC dose toxicity study in dogs was performed at doses of 0, 3, 8 and 20 mg/kg RT-104 HCl. Systemic exposures (Cₘₐₓ and AUC₀₋ₜ) of RE104 HCl and 4-OH-DiPT showed no sex differences. Increase in exposure was approximately dose-proportional for RE104 HCl and greater than dose-proportional for 4-OH-DiPT over the dose range 3 to 20 mg/kg. Clinical signs were limited to serotonin syndrome.

The NOAEL was judged to be 8 mg/kg in dog. The HED of 8 mg/kg in the dog is approximately 267 mg (expressed as zwitterion) assuming a 60 kg human body weight and standard BSA conversion factors. This HED provides a safety margin of approximately 53-fold relative to the proposed starting dose of 5 mg (expressed as zwitterion) and 5-fold relative to the proposed maximum dose of 48 mg (expressed as zwitterion), in this study. The 8 mg/kg dose in dogs was associated with a Cₘₐₓ and AUC₀₋ₜ of 3300 ng/mL and 4540 h·ng/mL, respectively for RE104, and 360 ng/mL and 1010 h·ng/mL, respectively for 4-OH-DiPT.

Based on a NOAEL of 10 mg/kg in rats, and a NOAEL of 8 mg/kg in dog, the maximum recommended safe starting dose (MRSD) of RE104 HCl for the RT-104-101 study, applying a 10-fold safety margin, is 9.7 mg (Table 1). The actual proposed starting dose for the first in human (FIH) study was 5 mg (RE104 free base) thus the safety margins relative to the starting dose were higher.

Based on comparison of the efficacious dose range for psilocybin/psilocin in various mental illness (21-30 mg orally), and taking in to account the oral bioavailability in man for psilocin of approximately 53%, and the affinity of psilocin at the 5-HT2A receptor, the effective dose for 4-OH-DiPT is estimated to be in the range of 10 mg to 30 mg. Subcutaneous dosing of RE-104 HCl in animals was 100% bioavailable, thus the parent dose is expected to be in the same dose range. Therefore, 5.5 mg (RE104 HCl fee base) was considered conservative and was expected to allow for a safe starting dose.

Based on the NOAEL and MRSD in Table 2, the proposed dose levels were proposed for some of the human Cohorts 1 to 4 scheduled for the RE104-101 study reported in Example 2.

### Example 2

### Study Rationale

The current study is the first clinical study to be conducted with RE104 HCl. It has been designed to investigate the safety, tolerability, PK and pharmacodynamic (PD) properties of RE104 HCl following single doses, administered as a SC injection in healthy women. The data obtained in this study will provide the basis for the further clinical development of RE104 HCl and will be used to support dose selection in future Phase 2 studies in the target patient population.

### Objective

To characterize the safety, tolerability, PD and PK of RE104 in a phase 1, first-in-human (FIH) study.

### Design

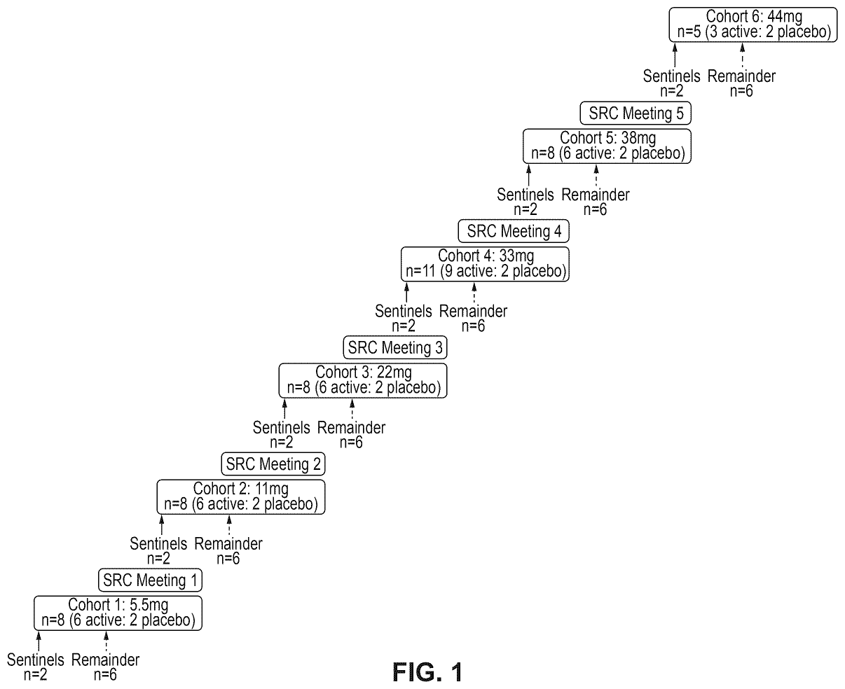

A schematic of the study design is set out in FIG. 1.

The study was a double-blind, randomized, placebo-controlled Phase 1 single ascending dose study in up to 6 cohorts in healthy females. Eligibility was assessed during a screening period of up to 28 days prior to enrollment. For the treatment period, subjects were admitted to the clinical research unit (CRU) one day prior to the dosing (Day −1). On Day 1, all subjects will received the assigned SC dose of RE104 HCl or placebo in a fed state (after domiciling overnight and consuming a planned evening meal on Day −1 and morning meal on Day 1 while at the CRU). Subjects were allowed to select snacks from a planned menu from 4 hours following dosing. Subjects were discharged from the clinic on Day 2 (−24 hours post-dose) after all required study procedures were completed and if deemed medically fit. Subjects returned to the clinic on Day 10 (±2 days) for a follow up visit.

Six (6) cohorts were enrolled in the study. Six (6) single doses were tested in 6 cohorts (Cohort 1 to Cohort 6) of 8 healthy female subjects (6 active and 2 placebo), as indicated in FIG. 1 and Table 3.

The starting dose was 5.5 mg RE104 HCl. Sentinels were used in all cohorts. Within each cohort, 2 subjects were treated first: 1 subject received RE104 HCl and 1 subject received placebo. Provided no clinically significant safety issues, were noted at least in the 24 hours following sentinels' dosing, as judged by the principal investigator (PI), the remaining 6 subjects of each cohort were dosed (5 active, 1 placebo).

The study was monitored by a safety review committee (SRC). The intent of the SRC is to ensure that treatment does not pose undue risk to subjects. Safety, tolerability, and available PK data were assessed by the SRC between each cohort before ascending to the next dose level (Table 3). After each cohort dosing was complete, the SRC met to discuss dose escalation. Escalation to the next higher dose level occurred only after evaluation of the safety and tolerability data up to and including at least 24 hours post-dose of all evaluable subjects in the previous dose cohort. Available PK results were requested for determination of dose escalation, as deemed necessary.

### Inclusion Criteria

The subject population included healthy adult subjects who satisfied all the following entry criteria:
Signed informed consent in a language understandable to the subject prior to any study-related procedure.
Healthy female subject aged between 18 and 65 years (inclusive) at screening.
The subject weighs at least 60 kg and has a BMI between 18.0 and 30.0 kg/m2 (inclusive) at screening.
The subject has self-reported at least one substantial prior recreational experience with hallucinogenic or psychedelic compounds (not including cannabis products) with the latest experience more than 90 days prior to study drug administration.
Women of childbearing potential (WOCBP) must be non-lactating and have a negative pregnancy test at screening and admission and be willing to have additional pregnancy tests as required throughout the study.
Women of childbearing potential (WOCBP) who are sexually active with a non-sterilized male partner must agree to consistently and correctly use a double-barrier highly effective method of contraception as assessed by the PI (for example a condom AND oral contraceptive pills [OCPs], long-acting implantable hormones, injectable hormones, a vaginal ring or an intrauterine device [IUD]) during the study and for at least 90 days after the study drug administration. If a hormonal contraceptive is used, it must have been initiated at least 30 days before study drug administration. Females who are abstinent from heterosexual intercourse as part of their usual lifestyle and are not planning to conceive will also be eligible for participation.
Females who are not WOCBP must be either surgically sterile (e.g., tubal occlusion, hysterectomy, bilateral salpingectomy, bilateral oophorectomy), or post-menopausal. Post-menopausal status will be confirmed through testing of follicle-stimulating hormone (FSH) levels (≥30 IU/mL) at screening for amenorrheic female subjects.
Female subjects must refrain from donating oocytes from screening up to at least 90 days after study drug administration.

### Exclusion Criteria

Subjects were excluded if they met any of the following criteria:
Subject has current or a history of any clinically significant illness, such as cardiovascular, neurologic, pulmonary, hepatic, renal, metabolic, gastrointestinal, urologic, immunologic, endocrine, or psychiatric disease or disorder, or other abnormality, which may affect safety, or potentially confound the study results.
Subject has current or a history of clinically significant mental disorders as assessed by a Mini International Neuropsychiatric Interview (MINI) questionnaire and interview by a qualified medical professional (clinical psychologist or psychiatrist) at screening.
Subject has a risk of suicide per the Columbia-Suicide Severity Rating Scale (C-SSRS) (a score of 4 or 5 on ideation or any suicidal behavior) at screening and baseline; or according to a qualified medical professional (clinical psychologist or psychiatrist) clinical judgment; or has a history of suicidal ideation or suicidal behavior.
Immediate (1st degree) blood-related family members, or personal currently or previously diagnosed with psychotic or bipolar disorder.
Any clinically significant illness, medical/surgical procedure, or trauma within 4 weeks prior to screening.
Subject has a history of cancer, except basal cell carcinoma which has been in remission for at least 5 years prior to screening.
Previous major adverse response to a hallucinogenic or psychedelic drug (not including cannabis products), as determined by the qualified/trained investigator.
Use of any medication, including over-the-counter (OTC) medication, in the 28 days or 5 half-lives (whichever is longer) before study drug administration, with the exception of:
Hormonal contraceptives for WOCBP.
Up to 3000 mg paracetamol per day, or up to 1200 mg ibuprofen per day for a maximum of three days.
Acute use of topical steroids.
Use of ayahuasca, kambó, yopo, ibogaine, psilocybin, dimethyltryptamine (DMT), 5-methoxy-N,N-dimethyltryptamine (5-MeO-DMT), lysergic acid diethylamide (LSD), Syrian Rue, or other psychedelic agents or mixtures in their synthetic or naturally-occurring form, and use of amphetamines, opioids, or 3,4-Methylenedioxymethamphetamine (MDMA), in the 90 days before screening and through to EOS/ET.
Use of synthetic or naturally-occurring cannabinoids from 28 days prior to study drug administration and agree not to use through to EOS/ET.
Subject has a positive cotinine at screening or on Day −1.
Positive alcohol breath test or urine screen for drugs of abuse at screening or on Day −1.
Subject has a history of drug abuse as per latest Diagnostic and Satistical Manual of Mental Dsorders (DSM) available at the beginning of the study or as determined by the medically qualified investigator or, within 1 year prior to screening.
Excessive alcohol consumption (regular alcohol intake ≥14 units per week for female subjects) within 180 days prior to screening. One unit (8 gr) is equivalent to ½ pint (280 mL) of beer, 1 measure (25 mL) of spirits, or 1 small glass (125 mL) of wine. Consumption of alcohol from 48 hours prior to study drug administration until last PK sample had been collected.
Consumption of products containing caffeine, xanthine or poppy seeds from 48 hours prior to study drug administration until last PK sample had been collected.
Participation in another clinical study involving study treatment within 30 days or 5 half-lives, whichever is longer, prior to screening.
Administration of any vaccine within 28 days prior to study drug administration and through to EOS/ET.
Use of any psychoactive medication (e.g., a selective serotonin reuptake inhibitor such as paroxetine or citalopram), haloperidol, any medication with MAO activity (such as isocarboxazid, phenelzine, selegiline or tranylcypromine, linezolid, and methylene blue), or any drug that has been indicated as a potential precipitative agent for serotonin syndrome within 28 days prior to study drug administration and through to EOS/ET. See Table 4 and Table 5, Section 5.3.1.1, for prohibited medications.
Any positive results for serum hepatitis B surface antigen (HbsAg), hepatitis C antibody and human immunodeficiency virus (HIV) at screening.
Female subjects who are pregnant, breastfeeding or have a positive pregnancy test at screening or on Day −1.
Resting (for at least 5 minutes) systolic blood pressure >140 or <90 mmHg or resting diastolic blood pressure >90 or <40 mmHg; Resting (for at least 5 minutes) pulse rate outside the range of <40 or >100 beats/min at screening or at any time point prior to study drug administration.
Any clinically significant abnormalities in rhythm, conduction, or morphology of the resting electrocardiogram (ECG), and any clinically important abnormalities in the 12-lead ECG as considered by the investigator that may interfere with the interpretation of Corrected QT interval (QTc) changes at screening or at any time point prior to study drug administration. QTc≥480 ms for a female subject at screening or Day −1.
The estimated glomerular filtration (eGFR) at screening is ≤60 ml/min/1.73 m.
The subject has any skin condition, abnormality of the administration site or tattoos that would preclude SC administration (e.g., local or systemic infection) and/or local site reactions assessments.
Subject has poor peripheral venous access.
Subject is an immediate family member, study site employee, or is in a dependent relationship with a study site employee who is involved in the conduct of this study (e.g., spouse, parent, child, sibling) or may consent under duress.
A positive test for severe acute respiratory syndrome coronavirus 2 (SARS-CoV-2; COVID-19) within 7 days of admission to the CRU.
Subject not fully vaccinated against COVID-19, as defined by local, state and national guidelines and requirements at the CRU.
Current evidence or history of COVID-19 or influenza-like illness as defined by fever (>37.5° C.) and 2 or more of the following symptoms within 7 days before dosing: cough, sore throat, runny nose, sneezing, limb/joint pain, headache, vomiting/diarrhea in the absence of a known cause, other than influenza or COVID-19 infection.

### Study Restrictions—Medication and Dietary Products

Table 4 indicates the list of prohibited medications and dietary products for the RE104-101 study. Table 5 reports a list of specific drugs that have the potential to precipitate serotonin syndrome and were prohibited for the study.

### Study Drug and Matching Placebo

The active study product was RE104 HCl, which is a pro-drug of the synthetic psychedelic drug 3-(2-diisopropylaminoethyl)-1H-indol-4-ol (4-OH-DiPT or isoprocin), a molecule structurally related to psilocin. 4-OH-DiPT acts as a 5-HT agonist, notably on 5-HT2A and 5-HT2B subtypes. RE104 HCl was used as a solution for injection. RE104 HCl was provided as 2 constituents: LYO-RE104 HCl, which contains a lyophilized form of the RE104 HCl and RE-PBS which is diluent, a phosphate buffered saline solution used as the diluent when preparing the final injection solution. Placebo was a volume matched 0.9/6 sterile saline solution.

Formulation, Storage. Preparation and Handling

RE104 HCl for SC injection used was a sterile lyophilized solid (LYO-RT-104 HCl) in a vial for reconstitution that was combined with an aqueous diluent, as per pharmacy manual. Each LYO-RE104 HCl vial contained an amount of the active ingredient that was equivalent to RE104 zwitterion.

For example, for Cohort 4, the vial contained 36.3 mg of lyophilized drug substance, equivalent to 33 mg of RE104 zwitterion. The diluent, which is composed of a near stoichiometric amount of sodium phosphate dibasic, was used to solubilize and neutralize the LYO-RE104 HCl, resulting in a solution of approximately pH 5 that is ready for injection. Before injection, 1.1 mL of RE-PBS was added to 36.3 mg vial of LYO-RE104 HCl to provide 33 mg/mL of RT-104 HCl. This is equivalent to 30 mg/mL of RE104 zwitterion (free base). The remaining Cohorts were handled in a correspondingly similar fashion.

LYO-RE104 HCl vials were stored at −20° C. RE-PBS, the diluent, was prepared aseptically, by combining water for injections, sodium phosphate dibasic (60 mM) and sodium chloride (40 mM) in solution. The composition was chosen so that sodium phosphate dibasic would neutralize the carboxylic acid of RE104 HCl, thus generating RE104 zwitterion in solution at pH 5. RE-PBS vials were stored at room temperature (15-25° C.). Once the RE104 and RE-PBS were combined, the solution was administered within 60 minutes.

### Dosing and Administration

Dosing took place at a time point agreed to with the investigational pharmacy and acceptable to the investigator. RE104 HCl or placebo were administered as a SC injection in the upper arm. Subjects received RE104 HCl or placebo in a fed state. The time of administration of RE104 HCl or placebo was be recorded in the eCRF. The regimen used is outlined in Table 3.

Within each cohort, 2 sentinel subjects were treated first: 1 subject received RE104 HCl and 1 subject received placebo. Provided no clinically significant safety issues, as judged by the PI, were noted at least in the 24 hours following sentinels' dosing, the remaining 6 subjects of the cohort were dosed (5 active, 1 placebo). All drug administration was be done under direct medical supervision. Study subjects were overseen during the potentially psychoactive study drug period by professionals trained to monitor psychedelic experiences.

### Safety and Tolerability

There were no serious adverse events (AEs) in any treated participants and all completed dosing (Table 6)

The most common treatment-related AEs for RE104 were nausea, sinus tachycardia (asymptomatic at a maximum recorded value of 115 beats per minute), restlessness, and headache.

Two participants, 1 at 38 mg and 1 at 44 mg, experienced distress as a severe AE and received midazolam. Both AEs resolved after administration of concomitant medication.

No injection site adverse reactions were reported, but there was 1 AE of mild bruising related to the administration procedure.

There were no evident blood pressure effects or clinically significant vital sign, clinical laboratory, or electrocardiogram findings during the study.

### Pharmacodynamics

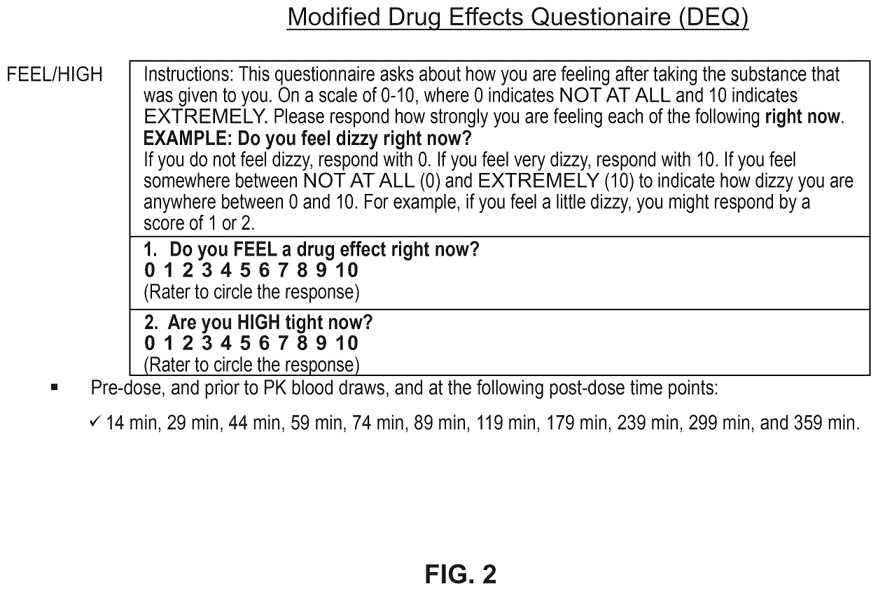

The Drug Effect Questionnaire (DEQ) Feel/High is widely used in studies of acute subjective response to a variety of substances, to assess two key aspects of subjective experience: (1) the strength of substance effects and (2) desirability of substance effects (Morean et al., 2013). The modified DEQ assesses the extent to which subjects (1) feel any substance effect(s) and (2) feel high. This version of the DEQ includes the following items: “Do you feel a drug effect right now?” (FEEL); “Are you high right now?” (HIGH). FIG. 2 sets out the modified DEQ that was used.

The modified DEQ was used as an 11-point numeric rating scale (0-10) administered by a qualified staff member. The subjects were asked to verbally respond to indicate how strongly they feel the drug effect and how high they feel on the scale of 0-10 after being administered the study drug (Kollltveit et al., 2020).

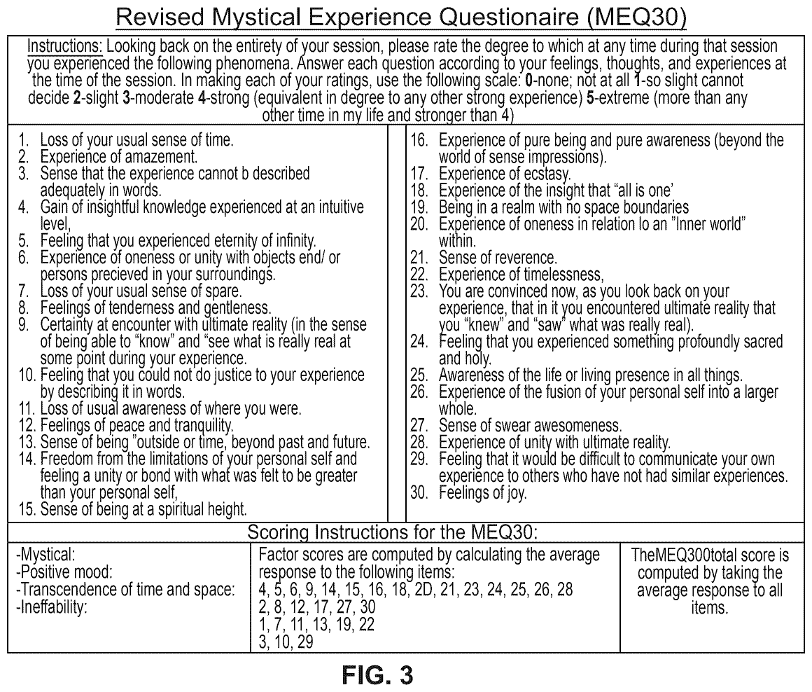

The Mystical Experience Questionnaire (MEQ30) is a validated questionnaire that assesses individual episodes of a mystical experience produced by classic hallucinogens (Barrett et al. 2015). The validated MEQ30 uses thirty questions across 4 areas of experience: (i) Mystical (including items concerning internal unity, external unity, noetic quality, and sacredness); (ii) Positive mood; (iii) Transcendence of time and space; and (iv) Ineffability. The subjects will be asked to rate each item of the MEQ30 on a 6-point scale. FIG. 3 sets out the revised MEQ30 that was used.

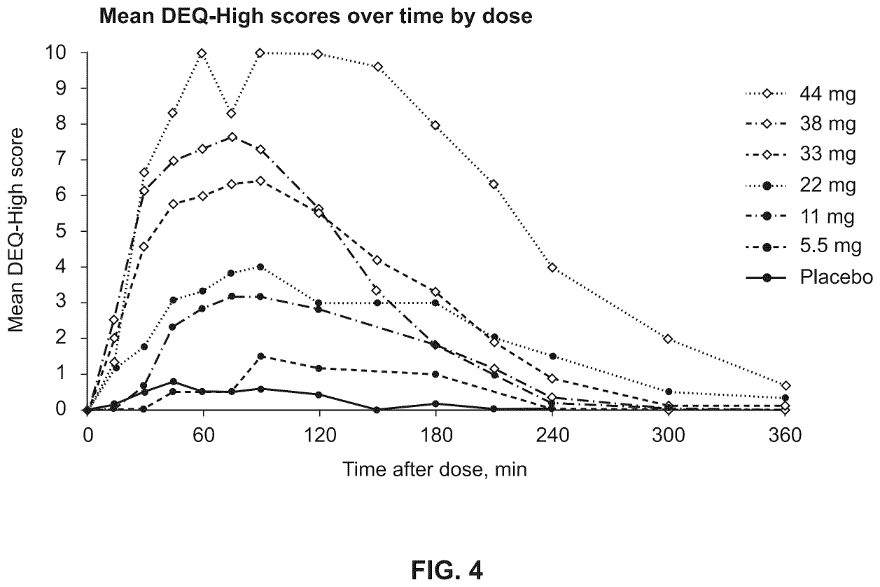

Average DEQ-High scores over time demonstrated a dose-dependent effect—see FIG. 4.

For doses 233 mg, peak DEQ-High scores ranged from 7 to 10 and the mean time to peak score was 1.1 hours; the Mean Experience Duration at 33 mg was 3.7 hours, and all participants had a score 51 at 4 hours post dose.

A dose-related increase in frequency of MEQ responders was observed, with 66.7%, 83.3%, and 100% of participants in the RE104 33-mg, 38-mg, and 44-mg treatment groups, respectively, having a “complete” mystical experience (defined as ≥60% of MEQ total score) predictive of clinical efficacy, based on MEQ total scores (Table 7)

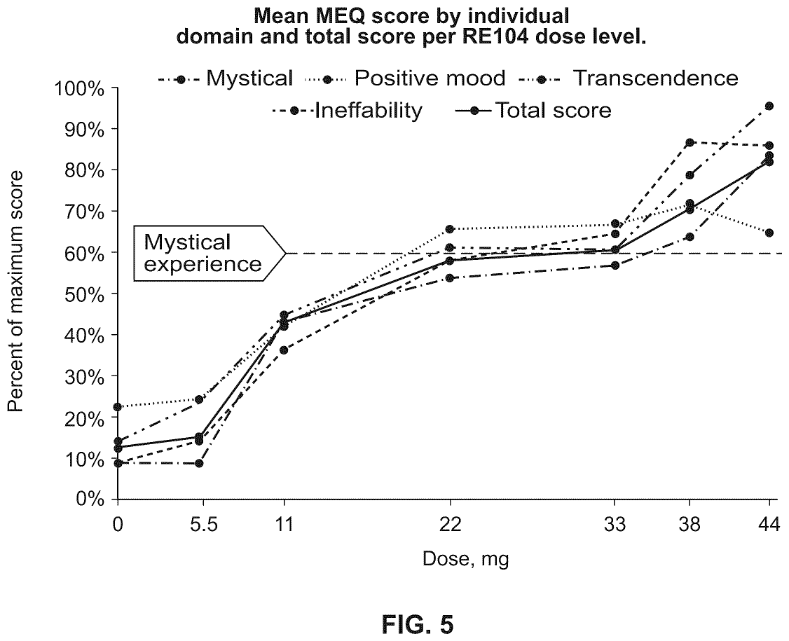

The proportion of MEQ responders by domain and dosing group is shown in FIG. 5.

### Pharmacokinetics

The concentrations of RE104 and isoprocin in plasma were determined using a validated Liquid chromatography tandem mass spectrometry (LC-MS/MS) assay.

The half-life of the RE104 prodrug was −0.5 hour across dose levels and RE104 was not detectable in plasma within 5 hours of administration at any dose.

The maximum plasma concentration of 4-OH-DiPT after administration of RE104 was proportional to the RE104 dose.

The results of PK testing are reported in Table 8.

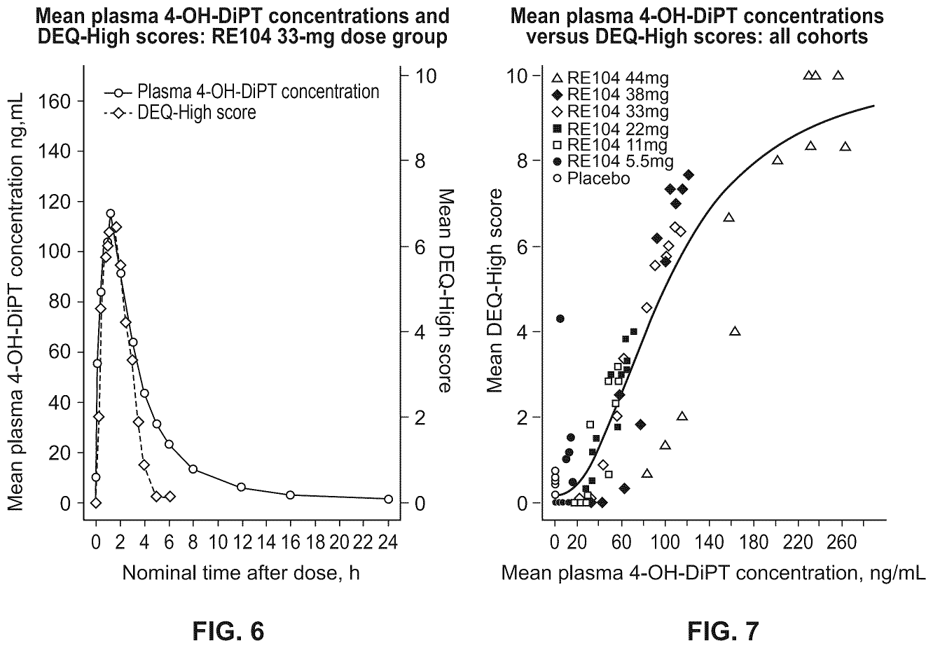

The PK profile of 4-OH-DiPT was aligned with the PD profile, with the peak plasma concentration and PD effects coinciding; however, the subjective drug effect declined faster than the plasma levels of 4-OH-DiPT (example from the 33-mg dose group shown in FIG. 6).

When PK-PD correlation was examined across all cohorts and dose levels, the mean 4-OH-DiPT concentration appeared to correlate with mean DEQ-High scores over a range of 40 to 100 ng/mL (FIG. 7).

Below 40 ng/mL, mean DEQ-High score did not exceed a value of 2.

### RE104 Phase 1 FIH Summary of Results

Overall, RE104 HCl was generally well tolerated with robust pharmacodynamic (PD) effects observed at doses 233 mg that closely aligned with the pharmacokinetic (PK) profile of 4-OH-DiPT.

The Drug Effect Questionnaire (DEQ-High) scores and Mystical Experience Questionnaire (MEQ) responder rates observed with RE104 doses ≥33 mg indicate potential for therapeutic effect in treatment trials, given that the intensity and quality of the subjective drug experience may predict treatment response to psilocybin therapy in depression (Griffiths et al.; Roseman et al.; and Ross et al.).

The mean duration of the subjective experience with RE104 HCl at 33 mg was 3.7 hours, representing a 50% reduced psychoactive experience relative to historical evidence with psilocybin, and a convenient duration for clinical monitoring.

The adverse effect (AE) profile of RE104 HCl is similar to psilocybin (Goodwin et al.; and Carbonaro et al.). with no serious AEs and no clinically significant vital sign, clinical laboratory, or electrocardiogram findings at doses up to and including 44 mg.

At doses ≥38 mg, 2 challenging experiences were observed.

These data informed dose selection of RE104 HCl at 33 mg for a planned randomized, active dose-controlled, phase 2 trial in women with moderate to severe postpartum depression (PPD).

Sub-cutaneous (SC) administration of the RE104 HCl at therapeutically relevant dose level (33 mg), demonstrates an improved safety and tolerability profile as compared to the administration of psilocybin; SC administration of the prodrug that allows for a faster absorption and earlier peak activity (approx 1 h), and shortest (optimal) duration of psychoactivity which is useful and conveneint to patient, physician and payer.

Administration of the pro-drug of RE104 HCl demonstrates improved pharacokitic profiles without excessive variability and with dose proportionality as compared to the administration of psilocybin.

Administration of the RE104 HCl demonstrates robust and pervasive pharmacodynamic effects at the 33 mg dose level with reduced duration of experience relative to psilocybin at 25 mg.

Administration of the pro-drug of RE104 HCl demonstrates an MEQ total score and domain scores reaching ≥60% for 33 mg, predictive of clinical efficacy as compared to the administration of psilocybin.

### Example 3

This Example describes the preclinical characterization of RE104, including pharmacologic, pharmacokinetic (PK), pharmacodynamic, and metabolic investigations. In anticipation of further clinical development as a potential treatment for depressive disorders, this Example reports the results of a 2-arm single-dose forced swim test (FST) study in rats over 28 days comparing the effects of RE104 and psilocin-4-glutarate (RE109), a psilocin prodrug also containing a glutarate moiety.

### Materials and Methods

### Molecule Synthesis

The synthesis of prodrugs RE104 and RE109 was carried pursuant to the teachings in U.S. Pat. No. 11,292,765B2 [Bryson].

### Receptor Pharmacology

Receptor binding and agonist activity (Eurofins Cerep, Celle-Lévescault, France) were determined as previously described (Bryant et al.; Choi et al,; and Porter et al.). Increasing concentrations of 4-OH-DiPT or RE104 were incubated with 0.1 nM [¹²⁵I]1-(4-iodo-2,5-dimethoxyphenyl)isopropylamine (DOI) in HEK-293 cell cultures expressing human recombinant 5-HT2A receptors. Increasing concentrations of RE104 were incubated with 0.2 nM [¹²⁵I]DOI in CHO cell cultures expressing human recombinant 5-HT2B receptors.

Radioligand concentration was measured by scintillation counting; compound binding was calculated as percent inhibition of the binding of [¹²⁵I]DOI. In agonist activity assays, test concentrations of RE104 were incubated with HEK-293 cells expressing human recombinant 5-HT2A receptors; IP1 production was detected using homogeneous time-resolved fluorescence. Activation was calculated as a percentage of the response to the positive control (10 μM 5-HT). Additional details are in the Supplemental Methods described below.

### Prodrug Cleavage

The stability of RE104 was assessed in 2 mL of plasma from male CD-1 mice, male Sprague Dawley rats, mixed-gender beagle dogs, and mixed-gender humans by measuring the concentration of RE104 and 4-OH-DiPT at 0, 0.5, 1, 2, and 4 hours of incubation at 37° C. using a liquid chromatography tandem mass spectrometry method (LC-MS/MS) (InterVivo Solutions, Inc, Mississauga, Ontario, Canada). Mean values from 3 replicate assays are reported. See the Supplemental Methods for additional details.

### Pharmacokinetics and Pharmacodynamics

Plasma samples from adult male Sprague Dawley rats were analyzed to determine RE104 and 4-OH-DiPT concentrations after RE104 intravenous (IV) dosing at 2 mg/kg (InterVivoSolutions, Inc). The concentration of test compounds was determined in plasma by LC-MS/MS. The dose of RE104 was kept at 2 mg/kg for subcutaneous (SC) and oral (PO) administration, whereas the dose of 4-OH-DiPT was adjusted to 1.39 mg/kg to match the molar dose (5.34 μmol/kg) of RE104. Pharmacokinetic parameters for each analyte and route of administration were estimated from the plasma concentration-versus-time curves for each animal. Head-twitch response (HTR), also referred to as wet dog shakes, was assessed by observing rats for automatisms consisting of rapid and alternating head rotations (González-Maeso et al. and Halberstadt et al.); HTRs were manually counted using 10-minute intervals up to 2 hours after dosing and again at 3, 3.5, and 4 hours after dosing.

Plasma PK was evaluated for RE104 and 4-OH-DiPT in male beagle dogs after 3 sequential doses via IV injection, SC injection, and oral gavage of RE104 at dose levels of 1, 1, and 5 mg/kg, respectively (QPS Taiwan, New Taipei City, Taiwan). Two consecutive doses were separated by z7 days. Plasma samples were analyzed using an LC-MS/MS method (lower limit of quantification of 1.00 ng/mL for both analytes).

Two male cynomolgus monkeys were administered 3 treatments in a crossover study design (Calvert Laboratories, Inc, Scott Township, PA; QPS, LLC, Newark, DE, USA). In treatment 1, RE104 was administered as an IV bolus dose at 1 mg/kg. In treatment 2, RE104 was administered as an SC injection at 0.5 mg/kg, and in treatment 3, RE104 was administered PO at 5 mg/kg, with all dosing formulations prepared in phosphate buffered saline. For each treatment, plasma PK of REJ04 and 4-OH-DiPT were analyzed via blood samples using an LC-MS/MS method.

Blood sampling time points are described in the Supplemental Methods. Pharmacokinetic parameters were calculated using Phoenix WinNonlin, version 6.3 or 8.2 (Certara, Princeton, NJ, USA).

### Metabolite Profiling and Identification

Metabolite profiling and identification was performed using rat, dog, and nonhuman primate plasma samples after a single SC injection of RE104 (QPS, LLC, Newark, DE, USA). Groups of 12 Sprague Dawley rats (6 male and 6 female, 8-10 weeks of age at dosing) received SC injections of RE104 at doses of 10, 25, or 75 mg/kg. Groups of 6 beagle dogs (3 male and 3 female, 9-10 months of age at dosing) each received SC injections of RE104 at 3, 8, or 20 mg/kg. Two male cynomolgus monkeys received a single SC injection of 0.5 mg/kg RE104.

Blood sampling timepoints are included in the Supplemental Methods. Blood samples were centrifuged to obtain plasma. Plasma samples for rat and dog studies were pooled across time points to 1 sample per dose and sex separately; plasma samples from monkeys were pooled to generate 1 combined sample.

In vitro metabolite profiling was performed using pooled (10 donors) cryoplateable primary human hepatocytes (QPS, LLC, Newark, DE, USA) incubated with 1 μM 4-OH-DiPT in triplicate or 1 μM lorazepam (positive control) in duplicate for up to 24 hours at 37° C. in a humidified incubator filled with 5% CO2. The formation of metabolites was monitored at 0, 4, 8, and 24 hours, respectively. For the negative control, 4-OH-DiPT at 1 μM was incubated in duplicate without hepatocytes. Samples for positive and negative controls were determined at 0, 8, and 24 hours, respectively. RE104 and its metabolites were identified and profiled using ultrahigh-performance liquid chromatography (Supplemental Methods).

### Forced Swim Test (FST)

The FST, a common model for antidepressant-like activity in rats that measures immobility, swimming, and climbing behaviors of rats after exposure to water, was adapted from a previously described psilocin protocol that applies standard FST observations up to 35 days after a single dose of psychedelic drug and revalidated using RE109 as the source of active silocin (Hibicke et al. and Slattery et al.).

Adult male Wistar rats (aged 8-10 weeks) were randomized in parallel to a single SC intraperitoneal injection dose of vehicle, 1 mg/kg RE104, or equivalent dose of RE-109 (1 mg/kg) (n=12 per treatment acclimatized to the experimental laboratory for 2 days and FST measurements were performed on days 7, 14, and 28. Each rat was placed in a water-filled cylinder (20 cm in diameter and 45 cm in height) for 15 minutes for initial exposure 24 hours before the test. On testing day, animals were placed individually in the glass cylinder containing 35 cm of water maintained at 25±1° C. A camera recorded the 5-minute session.

Immobility time, swimming time, and climbing time (in seconds) were measured for the 5-minute session from recorded videos by a blinded observer using a stopwatch. Statistical analysis was carried out with GraphPad Prism-5 using 1-way analysis of variance followed by Dunnett's test/Tukey's post hoc test or unpaired t test. For trend analyses, a linear repeated measures model was used for each endpoint. The intercept (model estimated time at dosing day), slope (change in swimming, climbing, and immobility time by study day), and P value for the slope parameter were calculated.

### Animal Welfare

All experiments involving animals were carried out according to protocols approved by the study facility's Institutional Animal Care and Use Committee or equivalent organization.

### Results

### Receptor Pharmacology

The chemical structures of the RE104 prodrug and the active compound 4-OH-DiPT have the following chemical structures:

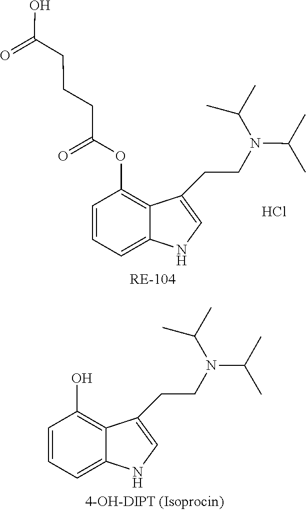 [SMILES: CC(C)N(CCc1c[nH]c2cccc(OC(=O)CCCC(=O)O)c12)C(C)C ; Cl ; CC(C)N(CCc1c[nH]c2cccc(O)c12)C(C)C]

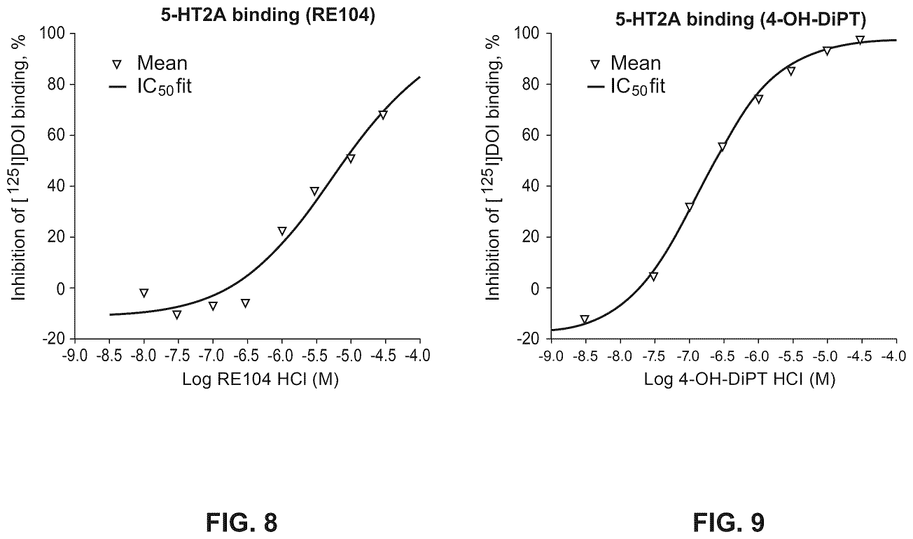

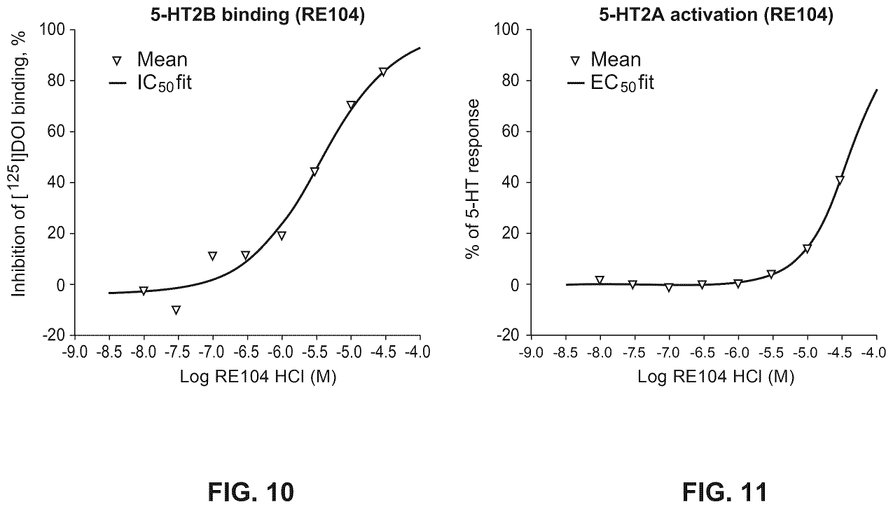

Binding affinities of RE104 and 4-OH-DiPT to the 5-HT2A receptor were determined using radioligand inhibition binding assays in HEK-293 cells. The binding Ki of RE104 and 4-OH-DiPT at the 5-HT2A receptor was 4300 nM (FIG. 8) and 120 nM (FIG. 9), respectively. Also evaluated was RE104 binding to the 5-HT2B receptor in CHO cells and the result obtained was a similar Ki to that of 5-HT2A receptor binding (1800 nM; FIG. 10).

To determine whether the RE104 prodrug is able to functionally activate 5-HT2A receptors, cellular agonist effect assays in HEK-293 cells were performed (FIG. 11). The EC50 of RE104 at the 5-HT2A receptor was >30,000 nM, indicating extremely low potency of RE104 at the 5-HT2A receptor.

### Prodrug Cleavage

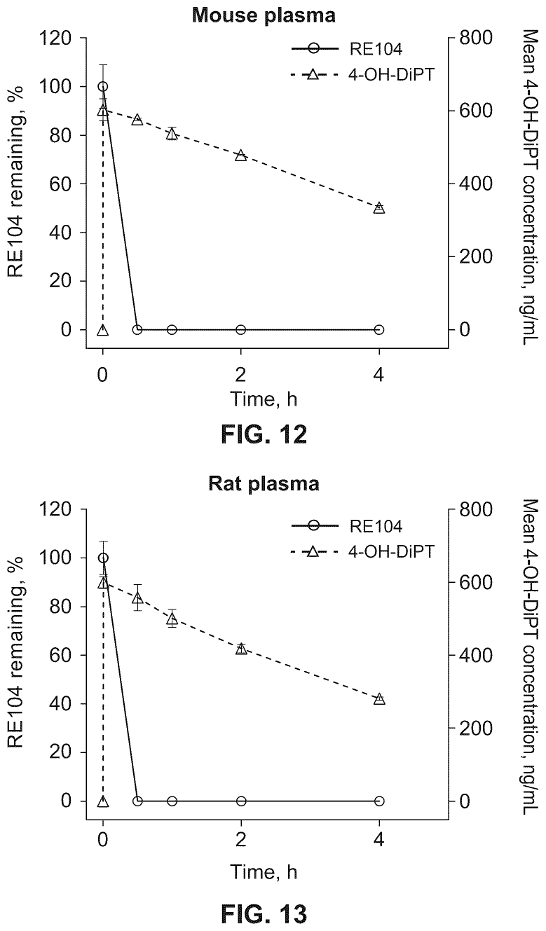

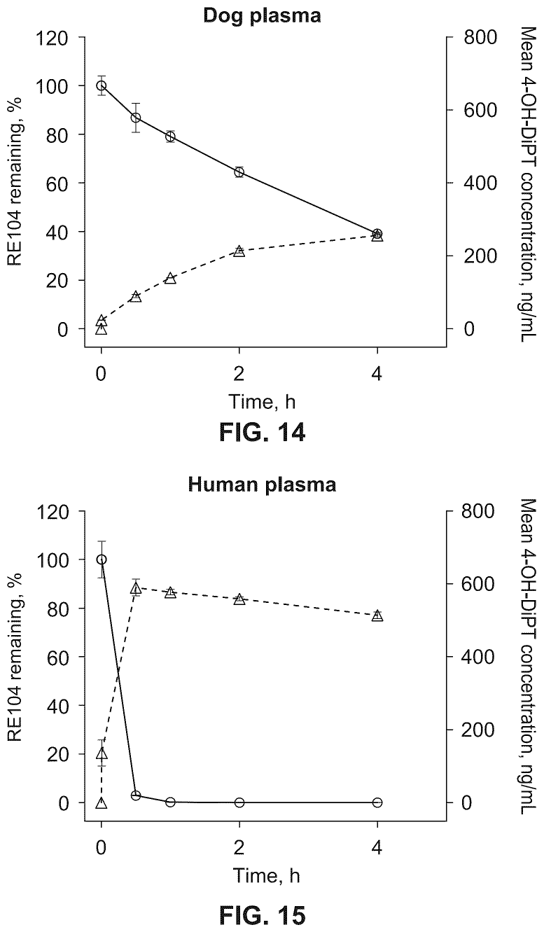

The kinetics of the conversion of the RE104 prodrug to active 4-OH-DiPT were measured in solution and plasma. Whereas RE104 demonstrated slow conversion in aqueous solution as a function of pH, conversion was rapid (<30 minutes) and complete (>95%) in plasma from mouse, rat, and human when incubated at 37° C. (FIGS. 12-15). Prodrug cleavage of RE104 was notably slower in dog plasma (39% remained after 4 hours of incubation). In all plasma matrices, 4-OH-DiPT was formed at rates and amounts consistent with hydrolysis of the glutarate leaving group of RE104; 4-OH-DiPT was oxidatively unstable in the media, as previously reported for the structurally related compound psilocin (Brown et al. and Hasler et al.). The psilocin prodrug RE109 demonstrated results similar to RE104 with rapid cleavage to psilocin when incubated with human plasma (data not shown).

Pharmacokinetic experiments were performed in rats, dogs, and nonhuman primates. Mean estimated PK parameters for 4-OH-DiPT after 2 mg/kg IV, PO, and SC administration of RE104 (prodrug) in Sprague Dawley rats are shown in Supplemental Table 1. After IV administration, RE104 was almost completely metabolized (96.7%) to 4-OH-DiPT within 5 minutes and was not measurable in plasma within 5 minutes of administration by any dosing method. The half-life of 4-OH-DiPT was similar after IV (0.60 hours) and SC (0.67 hours) administration of RE104. Overall mean plasma exposure (area under the curve [AUC]) for 4-OH-DiPT formed from RE104 was slightly higher after SC than after IV administration (AUC0-last [standard deviation]. 203 [42.8]h·ng/mL vs 146 [4.62]h·ng/mL), which may result from the higher concentration in the SC dosing solution (2.09 mg/mL) compared with the IV dosing solution (1.68 mg/mL) or variability associated with the low number of animals per treatment group (n=3). Overall plasma exposure of 4-OH-DiPT was extremely low after PO dosing, suggesting RE104 is poorly absorbed from the gastrointestinal tract in rats.

The PK of 4-OH-DiPT in rats was determined by direct administration of 4-OH-DiPT (1.39 mg/kg IV, PO, and SC) and mean estimated PK parameters for 4-OH-DiPT after administration of 4-OH-DiPT to rats are summarized in Supplemental Table 2. The smaller administered dose is equimolar to 2 mg/kg RE104, accounting for the smaller molecular weight of the active drug and allowing for direct comparability. When administered IV to rats, 4-OH-DiPT demonstrated a short half-life (0.74 hours) with a steady-state volume of distribution of 5.9 L/kg and systemic clearance of 9.4 L/h/kg. After SC administration, plasma concentrations peaked at 0.61 hour and mean bioavailability was 88.6%, whereas PO administration produced peak plasma concentrations at 0.25 hour and low mean bioavailability (0.825%).

The PK of RE104 and 4-OH-DiPT were determined after IV, SC, and PO administration in dogs. Consistent with in vitro cleavage studies, conversion of the prodrug to active drug was slower in dog than rat. RE104 was rapidly absorbed after SC (1 mg/kg) and PO (5 mg/kg) administration, and plasma concentrations exhibited rapid decline after IV bolus (Supplemental Table 3). The mean absolute bioavailability of RE104 was 121% after a single SC dose of 1 mg/kg RE104 and 0.854% after a single PO dose of 5 mg/kg RE104. The mean metabolic ratios of 4-OH-DiPT to RE104 were 0.339, 0.332, and 7.04 after a single dose of 1 mg/kg IV RE104, 1 mg/kg SC RE104, and 5 mg/kg PO RE104, respectively.

Similarly, RE104 was rapidly absorbed after IV (1 mg/kg) and SC (0.5 mg/kg) administration in nonhuman primates, with tₘₐₓ values 50.5 hours for 4-OH-DiPT plasma concentrations, indicating rapid conversion to 4-OH-DiPT; plasma 4-OH-DiPT was not quantifiable until 0.5 to 1 hour after oral dosing (5 mg/kg) of RE104. The mean terminal half-life of 4-OH-DiPT after IV and SC dosing was 0.63 hours and 0.95 hours, respectively. Taken together, these data confirm a <1-hour half-life of the active compound 4-OH-DiPT after IV and SC administration of RE104 in vivo and support SC dosing of RE104.

### Pharmacodynamics

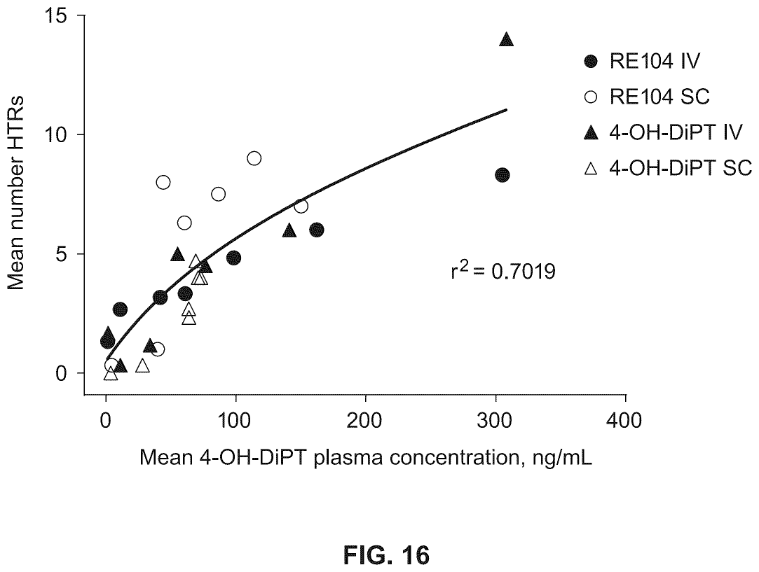

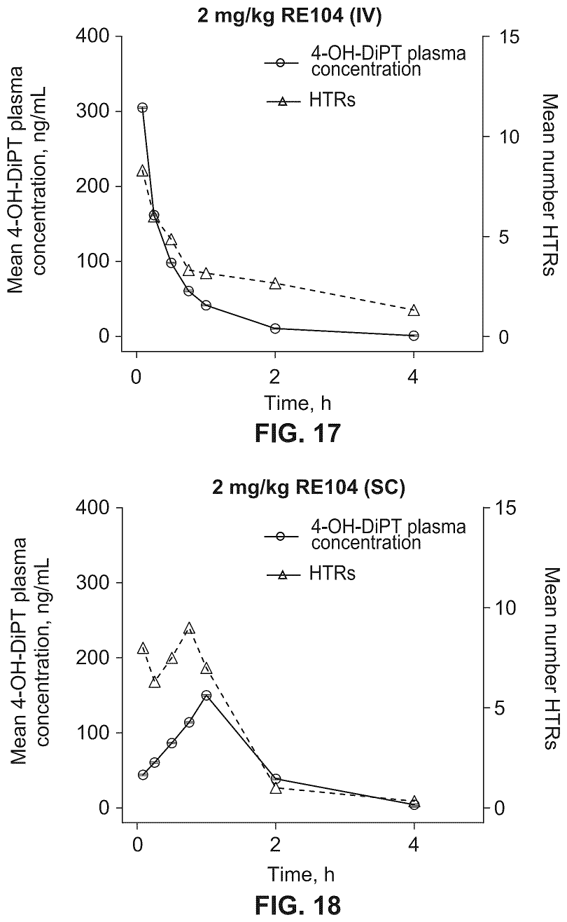

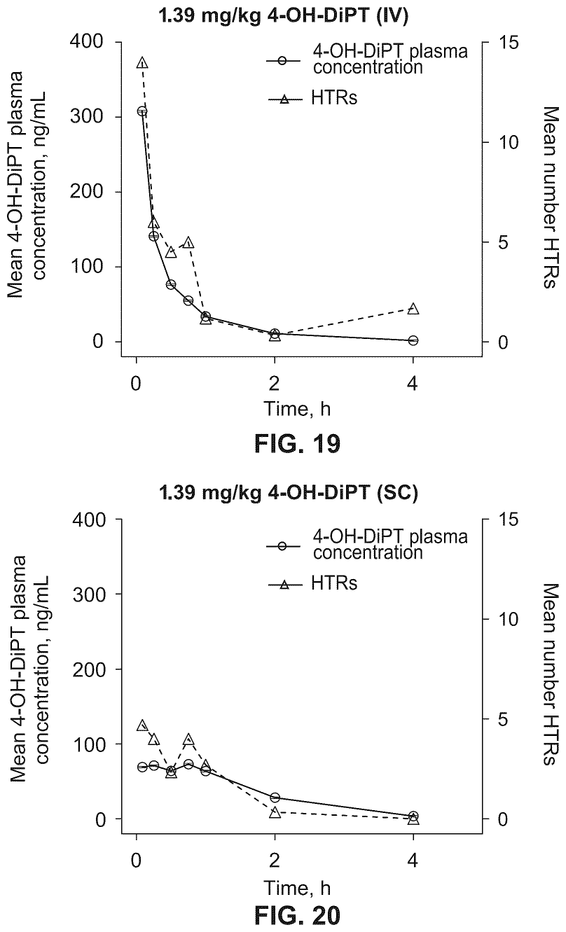

The HTR in rodents is a pharmacodynamic measure of 5-HT2A receptor agonism associated with psychedelic or hallucinatory effects (Gonzalez-Maeso et al. and Halberstadt et al. (2013)). To determine a potential PK-pharmacodynamic relationship, we measured HTRs after administration of RE104 and 4-OH-DiPT in rats. The mean number of HTRs over 10-minute intervals and the mean 4-OH-DiPT plasma concentrations in rats are plotted as a function of time in FIG. 16; HTR peaked immediately after IV administration of 2 mg/kg RE104 and 1.39 mg/kg 4-OH-DiPT and declined over the 4-hour observation period. After SC administration of 2 mg/kg RE104 or 1.39 mg/kg 4-OH-DiPT, mean HTR number peaked within 1 hour and similarly declined over the observation period. The HTR duration mirrored the PK profile of 4-OH-DiPT after IV and SC administration of 2 mg/kg RE104 and 1.39 mg/kg 4-OH-DiPT. Additionally, mean HTR values correlated with the 4-OH-DiPT plasma concentration after IV and SC administration of 2 mg/kg RE104 and 1.39 mg/kg 4-OH-DiPT (r²=0.7019; FIGS. 17-20).

### Metabolism

The metabolic profile of RE104 was determined across rats, dogs, and nonhuman primates in pooled plasma samples after a single SC dose of RE104. In 0- to 10-hour pooled plasma samples from rats administered 10, 25, or 75 mg/kg RE104, hydrolysis to 4-OH-DiPT represented the major metabolic pathway identified (>67% of total metabolites in plasma across all doses). The next most abundant metabolite in rat plasma was 4-hydroxy-indole-2-acetic acid (hydrolysis and N-dealkylation), representing up to −18% of total metabolites in pooled plasma samples across all doses of RE104. Other minor metabolic pathways (defined by <10% of the total peak area) in rat included hydrolysis followed by (1) oxidation, (2) double oxidation, (3) glucuronidation, (4) oxidation and glucuronidation, or (5) double oxidation, dehydrogenation, and glucuronidation.

The major metabolite in pooled dog plasma 0 to 12 hours after SC administration of 3, 8, and 20 mg/kg RE104 was 4-OH-DiPT, representing 3.34% to 16.5% of the total composition across all doses; a significant portion of the drug remained as RE104 (78.5% to 96.0% across all doses). Other metabolic pathways were minor and yielded multiple unidentified products, with molecular weights consistent with steps of single and multiple oxidation, glucuronidation, and hydrogenation. No other metabolite from this category achieved >3% of total plasma components across all doses.

In pooled plasma samples collected from monkeys at 0 to 12 hours post dose, unchanged RE104 parent molecule represented 71.1% of the total peak area, and the active compound 4-OH-DiPT represented 4.42% of the total peak area. The most abundant metabolite identified represented glucuronidation of 4-OH-DiPT (24.5% of total peak area). As rapid conversion to the active drug was anticipated, 1 μM 4-OH-DiPT was incubated with pooled plated human hepatocytes, primarily producing O-glucuronide and negligible amounts of other metabolites (including 4-hydroxy-indole-2-acetic acid), suggesting qualitatively similar metabolism of RE104 across test species.

### Antidepressant-Like Activity in the FST

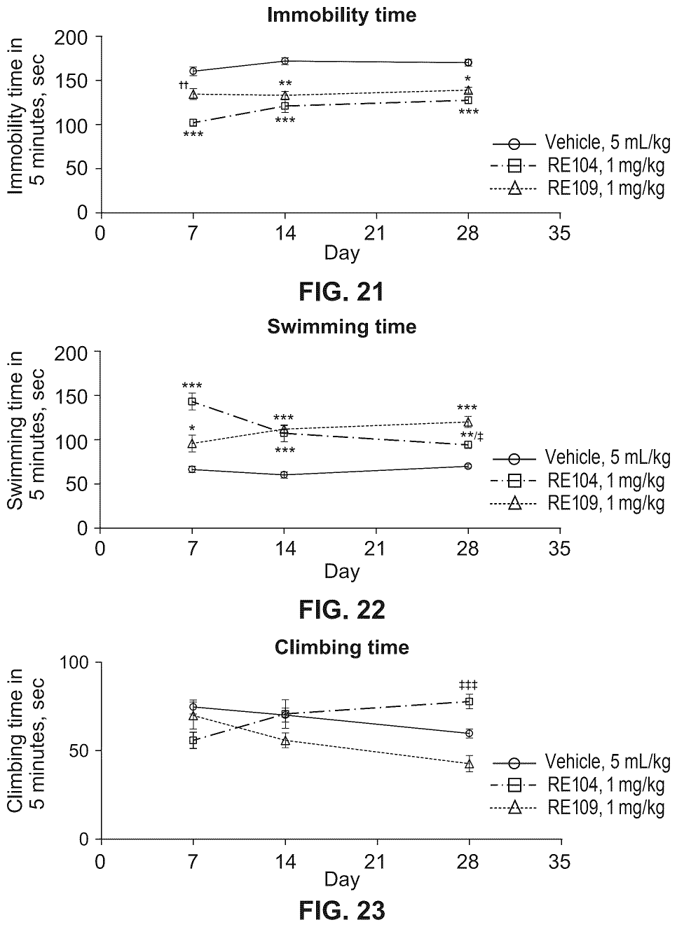

Antidepressant-like effects were evaluated using a modified FST in rats specifically developed to evaluate persistent effects after a single dose of psychedelic drugs (Hibicke et al.). When RE104 and RE109 were administered, distinctive serotonergic behaviors (e.g., HTR, piloerection, rearing, and flattened body posture) lasting approximately 3 hours were observed on the dosing day; these reactions were not observed in vehicle-treated animals. The first FST was performed 7 days after a single dose. A significant decrease in mean immobility time was observed for RE104 at day 7, 14, and 28, and for RE109 on day 14 and 28 (P<0.05; FIGS. 21-23), demonstrating persistent effects with both compounds.

Decreased immobility was accounted for by increased swimming and climbing escape behaviors (no diving was observed). Mean swimming time was significantly increased with both RE104 (P<0.01) and RE109 (P<0.05) relative to vehicle. Trends from day 7 to day 28 showed steadily increasing amounts of swimming for RE109 (P=0.08) with gradually decreasing amounts of climbing (P<0.004), whereas for RE104 climbing gradually increased (P<0.02) and swimming decreased (P<0.001) over the 28 days; immobility remained constant and different from vehicle for both compounds (P<0.05). By day 28, differences were observed between RE104 and RE109 with respect to the amount of swimming (P<0.05) and climbing (P<0.001), with swimming and climbing accounting for 55% and 45% of escape behaviors for RE104, respectively, versus 74% and 26% for RE109, respectively.

### Discussion

The results in the Example demonstrate that the active compound 4-OH-DiPT displays 5-HT2A receptor binding affinity with comparable magnitude to that previously described for the active compound of psilocybin (psilocin), supporting a similar pharmacologic profile (Rickli et al.). The prodrug RE104 prodrug is rapidly metabolized to 4-OH-DiPT in preclinical models and the half-life and PK profile of 4-OH-DiPT after RE104 administration corresponds with the duration of pharmacodynamic effects (HTR). Additionally, a single dose of RE104 in rats elicited prolonged antidepressant-like activity over 28 days in the FST comparable to that of psilocin formed in situ after administration of the psilocybin prodrug RE109. Collectively, these results confirm that RE104 administration most likely activates 5-HT2A receptors via the active compound 4-OH-DiPT to induce behavioral effects and antidepressant-like activity comparable to psilocybin (Gonzalez-Maeso et al. and Hibicke et al.).

The in vitro pharmacology data presented here are consistent with previous observations of 5-HT2A receptor binding of 4-OH-DiPT, with Ki values in the nanomolar range (Rickli et al.). Conversely, RE104 binding to 5-HT2A and 5-HT2B was substantially reduced compared with 4-OH-DiPT, with Ki values in the micromolar range. Functional activation of the 5-HT2A receptor by 4-OH-DiPT was previously reported with EC50 values of 6.82 nM and 93 nM (Rickli et al. and Gatch et al.). The EC50 for RE104 at 5-HT2A was >30,000 nM, demonstrating very low potency likely irrelevant for in vivo pharmacology. These results are also aligned with previous observations of 4-O-acetyl tryptamines having reduced activation of 5-HT2A compared with their 4-hydroxy counterparts (Klein et al.). Taken together, these results confirm 4-OH-DiPT is likely the only active species after administration of RE104.

The 4-OH-DiPT plasma concentration after RE104 administration correlates with pharmacodynamic HTR response in rats similar to PK-pharmacodynamic and pharmacodynamic-receptor occupancy correlations seen with psilocybin in human volunteers (Madsen et al.). The half-life of 4-OH-DiPT observed after IV or SC administration of RE104 in rats was approximately one-third that reported for psilocin after oral administration of Gymnopilus spectabilis, a mushroom species containing psilocin (terminal half-life for 4-OH-DiPT of approximately 40 minutes versus distribution and elimination half-lives for psilocin of 117 and 148 minutes, respectively) (Chen et al.). The PK and pharmacodynamic profiles observed are also consistent with anecdotal reports of the 2 to 3 hours duration of psychedelic effects of 4-OH-DiPT in humans (Shulgin), suggesting that administration of RE104 in a clinical setting might reduce clinical monitoring requirements compared with psilocybin administration (6-8 hours) (von Rotz et al.; Goodwin et al.; and Carbonaro et al.).

Taken together, the proposed distribution-metabolism model of RE104 is one in which the prodrug is enzymatically converted to 4-OH-DiPT. It is believed that circulating prodrug is peripherally restricted as a charged zwitterion at neutral pH and cannot readily diffuse into the brain. The active monoamine species can penetrate the central nervous system, causing nearly immediate but short-lived behavioral changes (e.g., HTR) and longer-term antidepressant-like behavior. Circulating 4-OH-DiPT will be metabolized to a glucuronide (major metabolite) or indole acetic acid (minor metabolite, rats), both likely inactive species that are eliminated. Generally, the metabolic profile of RE104 in animal models is similar to psilocybin and serotonin (Dinis-Oliveira), albeit the formation of indole acetic acid may be reduced in higher species (particularly humans) owing to difficulty of monoamine oxidase to metabolize the isopropyl derivative. In combination with metabolic profiling from human hepatocytes in vitro, these data support the elimination of RE104 primarily as a glucuronide metabolite most likely via urine and feces, again mirroring psilocybin (Dinis-Oliveira). Low absolute oral bioavailability of RE104 relative to IV or SC administration supports the use of an injectable formulation of RE104.

RE104 exhibited antidepressant-like activity similar to psilocin in an FST model of extended efficacy (Hibicke et al.). Both RE104 and RE109 reduced immobility time compared with vehicle, with effects maintained 28 days after a single dose. The findings with RE109 are consistent with previous reports of persistent antidepressant-like effects observed in the FST after single-dose psilocybin, and it is notable that these results have successfully translated to the clinic (Goodwin et al. and Hibicke et al.). Additionally, these data are consistent with prior reports that antidepressant-like effects of serotonergic agonists and reuptake inhibitors are specifically reflected by decreased immobility behavior in the FST, further suggesting that the antidepressant effects of serotonergic psychedelics are mediated by serotonergic modulation (Slattery et al. and Cryan et al.). While increased swimming and climbing accounted for the remaining test time for RE104 and RE109 administration, there were trends of increasing climbing over the 28-day period for RE104 and reduced climbing for RE109 that reached significance at day 28, whereas the complementary trend of increased swimming for RE109 and reduced swimming for RE104 was observed. Prior studies have shown that swimming and climbing behaviors are differentially responsive to selective serotonin reuptake inhibitors and selective norepinephrine reuptake inhibitors and are thus attributed to differential modulation of serotonergic and adrenergic systems, respectively (Detke et al and Lucki et al.). The preference for swimming over climbing responses in the FST has been observed with 5-HT1A agonists as well as in the original longitudinal FST study in rats administered psilocybin (Hibicke et al. and Lucki et al.); notably, psilocin is a stronger 5-HT1A agonist than 4-OH-DiPT (Rickli et al.). Moreover, climbing, not swimming, is the opposite of immobility (representing depression and/or despair) in rodents, when performed in a dosing paradigm with extended FST testing (Masuda et al.). The differences in climbing and swimming observed in the FST may indicate a small but durable difference in specific receptor system interactions and location and extent of downstream neuroplastic modifications that psychedelic molecules are capable of inducing (Rickli et al. and Detke et al.).

The results in this Example confirm that rapid cleavage of the novel prodrug RE104 to the 5-HT2A-activating compound 4-OH-DiPT results in pharmacodynamic effects indicative of psychedelic activity with short duration in preclinical models. Additionally, antidepressant-like activity of RE104 was confirmed in a translational proof-of-concept assay. Taken together, these studies demonstrate the potential for therapeutic effect of RE104 in humans in depressive disorders similar to that observed in recent randomized clinical trials of psilocybin, but with a reduced duration of the psychoactive state.

### Supplemental Methods

### Receptor Pharmacology

For the receptor binding assays, concentrations of test compounds were 3 nM, 30 nM, 100 nM, 300 nM, 1 μM, 3 μM, 10 μM, 30 μM (4-OH-DiPT), and 10 nM, 30 nM, 100 nM, 300 nM, 1 μM, 3 μM, 10 μM, and 30 μM (RE104). The half-maximal inhibitory concentration (IC50) and Hill coefficients were determined by nonlinear regression analysis of the competition curves generated with mean replicate (n=2) values using Hill equation curve fitting. Inhibition constants were calculated by using the Cheng-Prusoff equation Ki=IC50/(1+L/KD), where L=radioligand concentration and KD=radioligand affinity for the receptor (determined with a Scatchard plot). In the functional activity assay test concentrations of RE104 were 10 nM, 30 nM, 100 nM, 300 nM, 1 μM, 3 μM, 10 μM, and 30 μM. EC50 and IC50 values were determined by nonlinear regression analysis of the concentration-response curves generated with mean replicate values using Hill equation curve fitting. For all receptor pharmacology assays, data were analyzed with software developed at Cerep (Hill software, Celle-Lévescault, France) and validated by comparison with data generated by the commercial software SigmaPlot® 4.0 for Windows®(01997 by SPSS Inc).

### Prodrug Cleavage

Stock solutions of 0.1 mg/mL 4-OH-DiPT and RE104 were prepared in dimethyl sulfoxide stock solutions were diluted 100-fold to 1 μg/mL with 2 mL of pooled mixed-gender human, mixed-gender beagle dog, male CD-1 mouse, or male Sprague Dawley rat plasma (with K₂EDTA as anticoagulant). Triplicate aliquots (50 μL) of each spiked plasma solution were immediately added to 200 μL of ice-cold methanol/acetonitrile solution (50:50 v/v) containing the internal standards to generate time zero samples. Spiked plasma solutions were incubated at 37° C. in an orbital shaker set to 75 rpm, and 50-μL samples were taken in triplicate after 30 minutes and 1, 2, and 4 hours of incubation. Each sample was immediately added to 200 μL ofice-cold methanol/acetonitrile solution containing the internal standard(s) to terminate the reaction. Samples were mixed by vortex and then stored at −80° C. until analysis.

As RE104 was observed to be unstable in mouse, rat, and human plasma, calibration standards containing plasma could not be used for absolute quantification; therefore, the peak area ratios of the analyte/internal standard over incubation time are reported. For 4-OH-DiPT stability samples, mouse, dog, and human plasma samples were analyzed using rat plasma calibration standards. Calibration standards were prepared in plasma for each species and analyzed in batches by LC-MS/MS. Sample batches consisted of 3 replicates of a system suitability standard (containing the analyte and internal standard), the appropriate calibration standards in ascending order including a blank sample without internal standard, a zero sample (with internal standard), and at least 6 nonzero standards and the assay samples, followed by the 3 replicates of the system suitability sample.

### PK Sampling Methods and Timepoints

For rat PK assessments, approximately 0.25 mL blood was serially collected at 8 time points over 6 hours (5, 15, 30, and 45 minutes, and 1, 2, 4, and 6 hours after dosing) via a surgically placed carotid artery catheter and immediately transferred to tubes containing K₂EDTA on ice. For dog PK assessments, approximately 0.5 mL of blood was collected via superficial vein before dose and at 5 (for IV and SC injections only), 15, and 30 minutes and 1, 2, 4, 8, 16, 24, 48, and 72 hours post dose into tubes containing K2EDTA with 10 mM Dichlorvos as the anticoagulant and stabilizer, respectively. For nonhuman primate PK assessments, blood samples (˜1 mL) were collected via venipuncture of a femoral vein before dose and at 5, 15, and 30 minutes and 1, 1.5, 2, 4, 8, and 12 hours after dosing into tubes containing K2EDTA with 10 mM Dichlorvos.

Metabolite profiling and identificationFor experiments performed in rats, blood samples were collected before dose and at 15 minutes, 30 minutes, and 1, 2, 4, 6, and 10 hours after dose. Blood samples from dogs were collected before dose and at 5 minutes, 30 minutes, and 1, 2, 4, 8, 12, and 24 hours after dose. Monkey blood samples were collected before dose and at 5 minutes, 15 minutes, 30 minutes, and 1, 1.5, 2, 4, 8, and 12 hours after dose. A mixture of acetonitrile:methanol:for mic acid (50:50:0.1, v/v/v) at 3× the volume of plasma was added to the pooled plasma samples, mixed by vortex for 3 minutes, and centrifuged at 3000 rpm for 10 minutes at 4° C. Supernatant was evaporated in a 25° C. water bath under a stream of nitrogen gas. Dried residues were reconstituted in acetonitrile:methanol:water:formic acid (15:15:70:0.1, v/v/v/v) and centrifuged at 3000 rpm for 10 minutes before transfer into vials for ultrahigh-performance liquid chromatography (UHPLC).

For plated human hepatocytes, all samples were quenched with an equal amount of acetonitrile:methanol:for mic acid (50:50:0.1, v/v/v) after incubation with 4-OH-DiPT. The quenched samples were pipetting mixed, harvested, and stored at −70° C. or lower until liquid chromatography tandem mass spectrometry analysis. The processing for positive control lorazepam samples was identical to that of 4-OH-DiPT. The plated human hepatocyte incubation samples were frozen at ≤−70° C. or lower immediately and thawed to room temperature before sample processing. A 500-μL aliquot of each sample was transferred to matrix tubes (1.4 mL), followed by the addition of 500-μL mixed solvent acetonitrile:methanol:for mic acid (50:50:0.1, v/v/v). The resulting mixtures were vortex mixed for 2 minutes and centrifuged at 3000 rpm for 10 minutes. The supernatants from the triplicate incubations were pooled to generate the pooled samples (1.5 mL/each) for the LC-MS analysis.

For all metabolite identification and profiling experiments, RE104 and its metabolites were identified and profiled by UHPLC in a Shimadzu Nexeran™ UHPLC system coupled with a high-resolution mass spectrometer SCIEX 6600 Triple TOF®. Mass spectrometry analysis was carried out by using an electrospray ionization source operating in positive ion mode with full-scan TOF MS and MS/MS modes. Proposed metabolite structures were based on the accurate mass of the observed molecular ions and their associated mass fragmentation patterns. The fragmentation patterns of the proposed metabolites were compared with common fragment ions obtained from RE104 and 4-OH-DiPT reference standards. Metabolite Pilot software from Sciex was used to search for unknown metabolites. The molecular ions of potential metabolites were searched for based on metabolic pathways.

The percent of RE104 and its metabolites in relation to the total area of compound-related material was obtained by integration of the extracted ion chromatogram peaks on the full-scan mass-chromatograms. The percent of peak area for each component relative to the total peak area was determined by using the following formula: Component % peak area=(peak area of component ×100%/total integrated peak area of compound-related material).

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

### REFERENCES

Aday J S, Mitzkovitz C M, Bloesch E K, Davoli C C, Davis A K. Long-term effects of psychedelic drugs: A systematic review. Neurosci Biobehav Rev. 2020 June; 113:179-189. doi: 10.1016fj.neubiorev.2020.03.017. Epub 2020 Mar. 16. PMID: 32194129.
Andersen KAA, Carhart-Harris R, Nutt D J, Erritzoe D. Therapeutic effects of classic serotonergic psychedelics: A systematic review of modern-era clinical studies. Acta Psychiatr Scand. 2021 February; 143(2):101-118. doi: 10.1111/acps.13249. Epub 2020 Dec. 1. PMID: 33125716.
Barrett F S, Johnson M W, Griffiths R R. Validation of the revised Mystical Experience Questionnaire in experimental sessions with psilocybin. J Psychopharmacol. 2015 November; 29(11):1182-90. doi: 10.1177/0269881115609019. Epub 2015 Oct. 6. PMID: 26442957; PMCID: PMC5203697.
Bogenschutz M P, Forcehimes A A, Pommy J A, Wilcox C E, Barbosa P C, Strassman R J. Psilocybin-assisted treatment for alcohol dependence: a proof-of-concept study. J Psychopharmacol. 2015 March; 29(3):289-99. doi: 10.1177/0269881114565144. Epub 2015 Jan. 13. PMID: 25586396.
Boyer E W, Shannon M. The serotonin syndrome. N Engl J Med. 2005 Mar. 17; 352(11):1112-20. doi: 10.1056/NEJMra041867. Erratum in: N Engl J Med. 2007 Jun. 7; 356(23):2437. Erratum in: N Engl J Med. 2009 Oct. 22; 361(17):1714. PMID: 15784664.
Brown R T, Nicholas C R, Cozzi N V, Gassman M C, Cooper K M, Muller D, et al. Pharmaco-kinetics of escalating doses of oral psilocybin in healthy adults. Clin Pharmacokinet. 2017; 56:1543-54.
Bryant H U, Nelson D L, Button D, Cole H W, Baez M B, Lucaites V L, et al. A novel class of 5-HT2A receptor antagonists: aryl aminoguanidines. Life Sci. 1996; 59:1259-68.
Carbonaro T M, Johnson M W, Hurwitz E, Griffiths R R. Psychopharmacology (Berl). 2018:235(2):521-534.
Carhart-Harris R, Giribaldi B, Watts R, Baker-Jones M, Murphy-Beiner A, Murphy R, Martell J, Blemings A, Erritzoe D, Nutt D J. Trial of Psilocybin versus Escitalopram for Depression. N Engl J Med. 2021 Apr. 15; 384(15):1402-1411. doi: 10.1056/NEJMoa2032994. PMID: 33852780.
Carhart-Harris R L, Bolstridge M, Rucker J, Day C M, Erritzoe D, Kaelen M, Bloomfield M, Rickard J A, Forbes B, Feilding A, Taylor D, Pilling S, Curran V H, Nutt D J. Psilocybin with psychological support for treatment-resistant depression: an open-label feasibility study. Lancet Psychiatry. 2016 July; 3(7):619-27. doi: 10.1016/S2215-0366(16)30065-7. Epub 2016 May 17. PMID: 27210031.
Carhart-Harris R L, Erritzoe D, Williams T, Stone J M, Reed L J, Colasanti A, Tyacke R J, Leech R, Malizia A L, Murphy K, Hobden P, Evans J, Feilding A, Wise R G, Nutt D J. Neural correlates of the psychedelic state as determined by fMRI studies with psilocybin. Proc Natd Acad Sci USA. 2012 Feb. 7; 109(6):2138-43. doi: 10.1073/pnas.1119598109. Epub 2012 Jan. 23. PMID: 22308440; PMCID. PMC3277566.
Chernik D A, Gillings D, Laine H, Hendler J, Silver J M, Davidson A B, Schwam E M, Siegel J L. Validity and reliability of the Observer's Assessment of Alertness/Sedation Scale: study with intravenous midazolam. J Clin Psychopharmacol. 1990 August; 10(4):244-51. PMID: 2286697.
Chen J, Li M, Yan X, Wu E, Zhu H, Lee K J, et al. Determining the pharmacokinetics of psilocin in rat plasma using ultra-performance liquid chromatography coupled with a photodiode array detector after orally administering an extract of Gymnopilus spectabi-lis. J Chromatogr B Analyt Technol Biomed Life Sci. 2011; 879:2669-72.
Choi D S, Birraux G, Launay J M, Maroteaux L. The human serotonin 5-HT2B receptor: pharmacological link between 5-HT2 and 5-HT1D receptors. FEBS Lett. 1994; 352:393-9.
Cohen L B. Clinical trial: a dose-response study of fospropofol disodium for moderate sedation during colonoscopy. Aliment Pharmacol Ther. 2008 Apr. 1; 27(7):597-608. doi: 10.111 Ifj.1365-2036.2008.03598.x. Epub 2008 Jan. 10. PMID: 18194506.
Compass Therapeutics, COMP360 psilocybin therapy for treatment-resistant depression Phase fIb topline data, 9 Nov. 2021. COMP001 topline data (rocketcdn.me).
Cryan J F, Lucki I. Antidepressant-like behavioral effects mediated by 5-hydroxytryptamine2C receptors. J of Pharmacol Exp Ther. 2000; 295:1120-6.
Detke M J, Rickels M, Lucki I. Active behaviors in the rat forced swimming test differentially produced by serotonergic and noradrenergic antidepressants. Psychopharmacolo-gy (Berl). 1995; 121:66-72.
Diener E, Emmons R A, Larsen R J, Griffin S. The Satisfaction With Life Scale. J Pers Assess. 1985 February; 49(1):71-5. doi: 10.1207/s15327752jpa4901_13. PMID: 16367493.
Dinis-Oliveira R J. Metabolism of psilocybin and psilocin: clinical and forensic toxicological relevance. Drug Metab Rev. 2017; 49:84-91.
FDA Guidance for Industry. Estimating the maximum safe starting dose in initial clinical trials for therapeutics in adult healthy volunteers. Dated July 2005. URL: Guidance for Industry (fda.gov).
Gatch M B, Hoch A, Carbonaro T M. Discriminative Stimulus effects of substituted tryptamines in rats. ACS Pharmacol Transi Sci. 2021; 4:467-71.
Goodwin G M, Aaronson S T, Alvarez O, et al. N Engl J Med. 2022; 387(18):1637-1648.
González-Maeso J, Weisstaub N V, Zhou M, Chan P, Ivic L, Ang R, et al. Hallucinogens recruit specific cortical 5-H T(2A) receptor-mediated signaling pathways to affect behavior. Neuron. 2007,53:439-52.
Griffiths R R, Johnson M W, Carducci M A, Umbricht A, Richards W A, Richards B D, Cosimano M P, Klinedinst M A. Psilocybin produces substantial and sustained decreases in depression and anxiety in patients with life-threatening cancer: A randomized double-blind trial. J Psychopharmacol. 2016 December; 30(12):1181-1197. doi: 10.1177/0269881116675513. PMID: 27909165; PMCID: PMC5367557.
Griffiths R R, Johnson M W, Richards W A, Richards B D, McCann U, Jesse R. Psilocybin occasioned mystical-type experiences: immediate and persisting dose-related effects. Psychopharmacology (Berl). 2011 December; 218(4):649-65. doi: 10.1007/s00213-011-2358-5. Epub 2011 Jun. 15. PMID: 21674151; PMCID: PMC3308357.
Haberzettl R, Bert B, Fink H, Fox M A. Animal models of the serotonin syndrome: a systematic review. Behav Brain Res. 2013 Nov. 1; 256:328-45. doi: 10.1016/j.bbr.2013.08.045. Epub 2013 Sep. 1. PMID: 24004848.
Halberstadt A L, Koedood L, Powell S B, Geyer M A. Differential contributions of serotonin receptors to the behavioral effects of indoleamine hallucinogens in mice. J Psychopharmacol. 2011 November; 25(11):1548-61. doi: 10.1177/0269881110388326. Epub 2010 Dec. 8. PMID: 21148021; PMCID: PMC3531560.
Halberstadt A L, Geyer M A. Characterization of the head-twitch response induced by hallucinogens in mice: detection of the behavior based on the dynamics of head movement. Psychopharmacology (Berl). 2013; 227:727-39.
Hasler F, Bourquin D, Brenneisen R, Vollenweider F X. Renal excretion profiles of psilocin following oral administration of psilocybin: a controlled study in man. J Pharm Biomed Anal. 2002; 30:331-9.
Hibicke M, Landry A N, Kramer H M, Talman Z K, Nichols C D. Psychedelics, but not ketamine, produce persistent antidepressant-like effects in a rodent experimental system for the study of depression. ACS Chem Neurosci. 2020; 11:864-71.
https://www.uptodate.com/contents/serotonin-syndrome-serotonin-toxicity #H3, accessed on 5 Apr. 2022.
https://www.uspharmacist.com/article/drug-induced-serotonin-syndrome, accessed on 4 Apr. 2022.
ICH S7A Safety Pharmacology Studies for Human Pharmaceuticals. November 2000.
ICH S7B The Non-Ninical Evaluation of the Potential for Delayed Ventricular Repolarization (Q T Interval Prolongation) by Human Pharmaceuticals. May 2005.
International Council for Harmonisation (ICH), Integrated Addendum to ICH E6(R1): Guideline for Good Clinical Practice E6(R2), 9 Nov. 2016.
Investigator's Brochure, Version 1.0, dated 18 Feb. 2022.
Johnson M W, Garcia-Romeu A, Cosimano M P, Griffiths R R. Pilot study of the 5-HT2A R agonist psilocybin in the treatment of tobacco addiction. J Psychopharmacol. 2014 November; 28(11):983-92. doi: 10.1177/0269881114548296. Epub 2014 Sep. 11. PMID: 25213996; PMCID: PMC4286320.
Klein A K, Chatha M, Laskowski L J, Anderson El, Brandt S D, Chapman S J, et al. Inves-tigation of the structure-activity relationships of psilocybin analogues. ACS Pharma-col Transl Sci. 2021; 4:533-42.
Kollltveit J, Osaland M, Reimers M, Berle M. A comparison of pain registration by Visual Analog Scale and Numeric Rating Scale—a cross-sectional study of primary triage registration. medRxiv 2020.11.03.20225367; doi: https://doi.org/l0.1101/2020.11.03.20225367.
Kometer M, Schmidt A, Bachmann R, Studerus E, Seifritz E, Vollenweider F X. Psilocybin biases facial recognition, goal-directed behavior, and mood state toward positive relative to negative emotions through different serotonergic subreceptors. Biol Psychiatry. 2012 Dec. 1; 72(11):898-906. doi: 10.1016/j.biopsych.2012.04.005. Epub 2012 May 10. PMID: 22578254.
Lucki I. The forced swimming test as a model for core and component behavioral effects of antidepressant drugs. Behav Pharmacol. 1997; 8:523-32.
Madsen M K, Fisher P M, Burmester D, Dyssegaard A, Stenbmk D S, Kristiansen S, et al. Psychedelic effects of psilocybin correlate with serotonin 2A receptor occupancy and plasma psilocin levels. Neuropsychopharmacology. 2019; 44:1328-34.
Meltzer H Y. Role of serotonin in depression. Ann N Y Acad Sci. 1990; 600:486-99; discussion 499-500. doi: 10.1111/j.1749-6632.1990.tbl6904.x. PMID: 2252328.
Morean M E, de Wit H, King A C, Sofuoglu M, Rueger S Y, O'Malley S S. The drug effects questionnaire: psychometric support across three drug types. Psychopharmacology (Berl). 2013 May; 227(1):177-92. doi: 10.1007/s00213-012-2954-z. Epub 2012 Dec. 28. PMID: 23271193; PMCID: PMC3624068.
Moreno F A, Wiegand C B, Taitano E K, Delgado P L. Safety, tolerability, and efficacy of psilocybin in 9 patients with obsessive-compulsive disorder. J Clin Psychiatry. 2006 November; 67(11):1735-40. doi: 10.4088/jcp.v67n1110. PMID: 17196053.
Nichols D E. Hallucinogens. Pharmacol Ther. 2004 February; 101(2):131-81. doi: 10.1016/j.pharmthera.2003.11.002. PMID: 14761703.
Pambianco D J, Vargo J J, Pruitt R E, Hardi R, Martin J F. Computer-assisted personalized sedation for upper endoscopy and colonoscopy: a comparative, multicenter randomized study. Gastrointest Endosc. 2011 April; 73(4):765-72. doi: 10.1016/j.gie.2010.10.031. Epub 2010 Dec. 18. PMID: 21168841.
Porter R H, Benwell K R, Lamb H, Malcolm C S, Allen N H, Revell D F, et al. Functional characterization of agonists at recombinant human 5-HT2A, 5-HT2B and 5-HT2C receptors in CHO-K1 cells. Br J Pharmacol. 1999; 128:13-20.
Repke D B, Ferguson W J, Bates D K. Psilocin analogs. 1. Synthesis of 3-[2-(dialkylamino) ethyl]- and 3-[2-(cycloalkylamino) ethyl]indol-4-ols. J Heterocycl Chem. 1977; 14:71-4.
Rickli A, Moning O D, Hoener M C, Liechti M E. Receptor interaction profiles of novel psychoactive tryptamines compared with classic hallucinogens. Eur Neuropsycho-pharmacol. 2016; 26:1327-37.
Roseman L, Nutt D J, Carhart-Harris R L. Front Pharmacol. 2018; 8:974.
Ross S, Bossis A, Guss J, Agin-Liebes G, Malone T, Cohen B, Mennenga S E, Belser A, Kalliontzi K, Babb J, Su Z, Corby P, Schmidt B L. Rapid and sustained symptom reduction following psilocybin treatment for anxiety and depression in patients with life-threatening cancer: a randomized controlled trial. J Psychopharmacol. 2016 December; 30(12):1165-1180. doi: 10.1177/0269881116675512. PMID: 27909164; PMCID: PMC5367551.
Ross S, Bossis A, Guss J, Agin-Liebes G, Malone T, Cohen B, Mennenga S E, Belser A, Kalliontzi K, Babb J, Su Z, Corby P, Schmidt B L. Rapid and sustained symptom reduction following psilocybin treatment for anxiety and depression in patients with life-threatening cancer: a randomized controlled trial. J Psychopharmacol. 2016 December; 30(12):1165-1180. doi: 10.1177/0269881116675512. PMID: 27909164; PMCID: PMC5367551.
Sheehan D V, Lecrubier Y, Sheehan K H, Amorim P, Janavs J, Weiller E, Hergueta T, Baker R, Dunbar G C. The Mini-International Neuropsychiatric Interview (M.I.N.I.): the development and validation of a structured diagnostic psychiatric interview for DSM-IV and ICD-10. J Clin Psychiatry. 1998; 59 Suppl 20:22-33; quiz 34-57. PMID: 9881538.
Shulgin A, Shulgin A. Tihkal: The Continuation. Transform Press: Berkeley, C A; 1997.
Swift T C, Belser A B, Agin-Liebes G, et al. Cancer at the Dinner Table: Experiences of Psilocybin-Assisted Psychotherapy for the Treatment of Cancer-Related Distress. Journal of Humanistic Psychology. 2017; 57(5):488-519. doi:10.1177/0022167817715966.
Slattery D A, Cryan J F. Using the rat forced swim test to assess antidepressant-like activity in rodents. Nat Protoc. 2012; 7:1009-14.
Vollenweider F X, Vollenweider-Scherpenhuyzen M F, Bäbler A, Vogel H, Hell D. Psilocybin induces schizophrenia-like psychosis in humans via a serotonin-2 agonist action. Neuroreport. 1998 Dec. 1; 9(17):3897-902. doi: 10.1097/00001756-199812010-00024. PMID: 9875725.
Vollenweider F X. Brain mechanisms of hallucinogens and entactogens. Dialogues Clin Neurosci. 2001 December; 3(4):265-79. doi: 10.31887/DCNS.2001.3.4/fxvollenweider. PMID: 22033605; PMCID: PMC3181663.
von Rotz R, Schindowski E M, Jungwirth J, Schuldt A, Rieser N M, Zahoranszky K, et al. Single-dose psilocybin-assisted therapy in major depressive disorder: A placebo-controlled, double-blind, randomised clinical trial. EClinicalMedicine. 2023; 56:101809.
Willins D L, Meltzer H Y. Direct injection of 5-HT2A receptor agonists into the medial prefrontal cortex produces a head-twitch response in rats. J Pharmacol Exp Ther. 1997 August; 282(2):699-706. PMID: 9262333.

### Definitions and Interpretation

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated. To the extent that the following description is of a specific embodiment or a particular use of the invention, it is intended to be illustrative only, and not limiting of the claimed invention.

The corresponding structures, materials, acts, and equivalents of all means or steps plus function elements in the claims appended to this specification are intended to include any structure, material, or act for performing the function in combination with other claimed elements as specifically claimed.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to combine, affect or connect such aspect, feature, structure, or characteristic with other embodiments, whether or not such connection or combination is explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an element, item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all ranges described herein, and all language such as “between”, “up to”, “at least”, “greater than”, “less than”, “more than”, “or more”, and the like, include the number(s) recited and such terms refer to ranges that can be subsequently broken down into sub-ranges as discussed above.

### LIST OF EMBODIMENTS

The following is a list of non-limiting embodiments of the present invention:
1. An injectable composition comprising a compound of Formula (I):

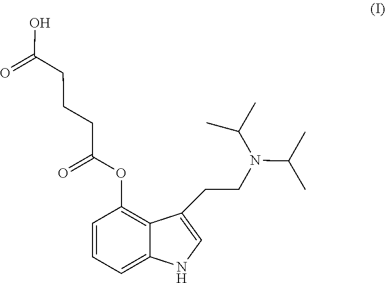 [SMILES: CC(C)N(CCc1c[nH]c2cccc(OC(=O)CCCC(=O)O)c12)C(C)C]

or a pharmaceutically acceptable salt thereof, wherein the compound of Formula (I) is present at a dose of from about 30 mg to about 50 mg calculated as the free base, together with a pharmaceutically acceptable carrier.
2. An injectable composition comprising a compound of Formula (I):

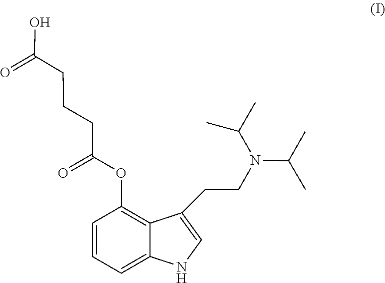 [SMILES: CC(C)N(CCc1c[nH]c2cccc(OC(=O)CCCC(=O)O)c12)C(C)C]

or a pharmaceutically acceptable salt thereof, wherein the compound of Formula (I) is present at a dose of 30 mg or 40 mg calculated as the free base, together with a pharmaceutically acceptable carrier.
3. The injectable composition defined in Embodiment 1 or Embodiment 2, wherein the compound of Formula (I) is the hydrochloride salt thereof
4. The injectable composition defined in Embodiment 2, wherein the compound of Formula (I) is the hydrochloride salt thereof present at a dose of 33 mg or 44 mg.
5. The injectable composition defined in any one of Embodiments 1-4, wherein wherein the injectable composition is a subcutaneous injectable composition.
6. A method of treating a mental disorder comprising the step of administering to a patient in need of treatment a pharmaceutical composition comprising a compound of Formula (I):

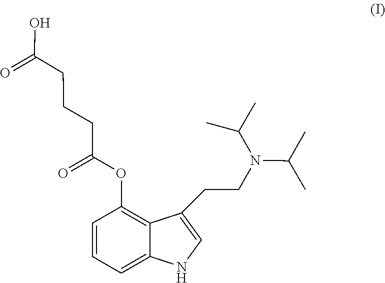 [SMILES: CC(C)N(CCc1c[nH]c2cccc(OC(=O)CCCC(=O)O)c12)C(C)C]

at a dose of from about 30 mg to about 50 mg calculated as the free base, together with a pharmaceutically acceptable carrier.
7. A method of treating a mental disorder comprising the step of administering to a patient in need of treatment a pharmaceutical composition comprising a compound of Formula (I):

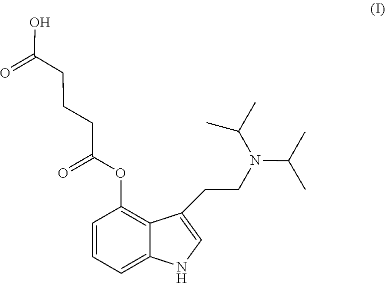 [SMILES: CC(C)N(CCc1c[nH]c2cccc(OC(=O)CCCC(=O)O)c12)C(C)C]

at a dose of about 30 mg or about 40 mg calculated as the free base, together with a pharmaceutically acceptable carrier.
8. The method defined in Embodiment 6 or Embodiment 7, wherein the compound of Formula (I) is the hydrochloride salt thereof.
9. The method defined in Embodiment 7, wherein the compound of Formula (I) is the hydrochloride salt thereof present at a dose of 33 mg or 44 mg.
10. A method of treating a mental disorder comprising the step of administering to a patient in need of treatment a pharmaceutical composition comprising a compound of Formula (I):

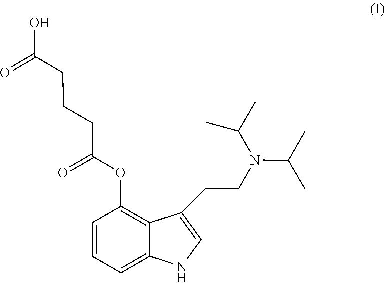 [SMILES: CC(C)N(CCc1c[nH]c2cccc(OC(=O)CCCC(=O)O)c12)C(C)C]

at a dose of 1 mg/kg calculated as the free base, together with a pharmaceutically acceptable carrier.
11. The method defined in any one of Embodiments 6-10, wherein the compound of Formula (I) has a duration of action of from about 2.5 hours to about 4.5 hours after administration.
12. The method defined in any one of Embodiments 6-10, wherein the compound of Formula (I) has a duration of action of about 3.5 hours or 3.7 hours after administration.
13. The method defined in any one of Embodiments 6-12, wherein the compound of Formula (I) has a Cₘₐₓ in the patient in the range of from about 1000 ng/mL to about 2000 ng/mL.
14. The method defined in any one of Embodiments 6-12, wherein the compound of Formula (I) has a Cₘₐₓ in the patient in the range of from about 1500 ng/mL to about 1800 ng/mL
15. The method defined in any one of Embodiments 6-14, wherein the compound of Formula (I) has a Tₘₐₓ in the patient in the range of from about 20 minutes 30 minutes.
16. The method defined in any one of Embodiments 6-14, wherein the compound of Formula (I) has a Tₘₐₓ in the patient in the range of about 15 minutes.
17. The method defined in any one of Embodiments 6-16, wherein the compound of Formula (I) is converted to a compound of Formula (II) after administration

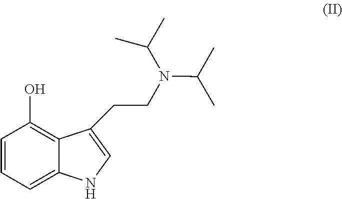 [SMILES: CC(C)N(CCc1c[nH]c2cccc(O)c12)C(C)C]

and the compound of Formula (II) has a Cₘₐₓ in the patient in the range of from about 100 ng/mL to about 300 ng/mL.
18. The method defined in any one of Embodiments 6-16, wherein the compound of Formula (I) is converted to a compound of Formula (II) after administration

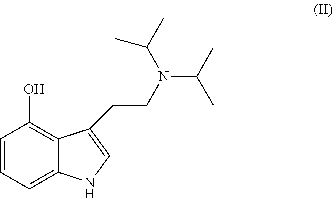 [SMILES: CC(C)N(CCc1c[nH]c2cccc(O)c12)C(C)C]

and the compound of Formula (II) has a Cₘₐₓ in the patient in the range of from about 120 ng/mL to about 300 ng/mL.
19. The method defined in any one of Embodiments 6-18, wherein the compound of Formula (I) is converted to a compound of Formula (II) after administration

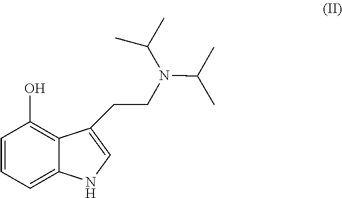 [SMILES: CC(C)N(CCc1c[nH]c2cccc(O)c12)C(C)C]

and the compound of Formula (II) has a Tₘₐₓ in the patient in the range of from about 60 minutes to about 150 min.
20. The method defined in any one of Embodiments 6-18, wherein the compound of Formula (I) is converted to a compound of Formula (II) after administration

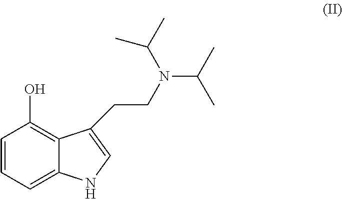 [SMILES: CC(C)N(CCc1c[nH]c2cccc(O)c12)C(C)C]

and the compound of Formula (II) has a Tₘₐₓ in the patient in the range of from about 60 minutes to about 80 min.
21. The method defined in any one of Embodiments 6-20, which demonstrates an average MEQ30 score in the patient of at least 50%.
22. The method defined in any one of Embodiments 6-20, which demonstrates an average MEQ30 score in the patient of at least 60%.
23. The method defined in any one of Embodiments 6-22, wherein the pharmaceutical composition is an injectable pharmaceutical composition.
24. The method defined in any one of Embodiments 6-22, wherein the pharmaceutical composition is an subcutaneous injectable pharmaceutical composition.

TABLE 1
Maximum Recommended Safe Starting Dose (MRSD) of RE104 (zwitterion)

| | | | | | | | 1/10 of | |
|---|---|---|---|---|---|---|---|---|
| | | | | | Human | Human | Human | |
| | RE -104 | | 1/10 of | 1/10 of | BSA | BSA | BSA | |
| | NOAEL | HED^{a} | HED | HED | Dose | dose | dose | MRSD |
| Species | (mg/kg) | (mg/kg) | (mg/kg) | (nag)^{b} | (mg/m²) | (mg)^{c} | (mg) | (mg) |
| Rat | 10^{d} | 1.6 | 0.16 | 9.6 | 60 | 97.2 | 9.7 | 9.7 |
| Dog | 8^{e} | 4.4 | 0.44 | 26.4 | 160 | 259 | 25.9 | |

Abbreviations: BSA = body surface area; HED = human equivalent dose; NOAEL = no observed adverse effect level; MRSD = maximum recommended safe starting dose.
^{a}HED calculated based on BSA conversion factors from animals to humans as detailed in FDA guidance, 2005.
^{b}Based on a 60 kg body weight.
^{c}Based on a BSA of 1.62 m².
^{d}NOAEL = 10 mg/kg in female rats
^{e}NOAEL = 8 mg/kg in female and male dogs.

TABLE 2
Proposed dose levels Cohorts 1-4

| | Proposed RE104 | Fold Lower than Scaled |
|---|---|---|
| Cohort | HCl Dose (mg)^{a} | NOAEL Dose^{b} |
| 1 | 5.5 | 19 |
| 2 | 11 | 9.7 |
| 3 | 22 | 4.9 |
| 4 | 33 | 3.2 |

^{a}Doses are shown m the table as RE104 HCl. These doses are equivalent to 5, 10, 20, and 30 mg RE104-zwitterion.
^{b}Example calculation for Cohort 1: 97.2 mg/5 mg = 19-fold lower than the scaled NOAEL dose.

TABLE 3
Dosing Regimen for the RE104-101 Study

| | | Number Subjects | Number Subjects |
|---|---|---|---|
| Cohort | RE104 HCl | to Be Treated | to Be Treated |
| Number | Dosing (mg) | With RE- 104 HCl | With Placebo |
| 1 | 5.5 | 6 | 2 |
| 2 | 11 | 6 | 2 |
| 3 | 22 | 6 | 2 |
| 4 | 33 | 9 | 2 |
| 5 | 38 | 6 | 2 |
| 6 | 44 | 3 | 2 |

TABLE 4
Prohibited Medications

| 90 Days Prior to Drug | 28 Days (or from screening or | |
|---|---|---|
| Administration (Day 1) | 5 Half- Life as | 48 Hours Prior to Study Drug |
| to the End of | Applicable) Prior to Study Drug | Administration Until Last PK |
| the EOS/ET Visit | Administration to the EOS/ET Visit | Sample Has Been Collected |
| Use of ayahuasca, kambo, | Prescription medications/OTC | Products containing |
| yopo, ibogaine, psilocybin, | medications in the 28 days or 5 half- | caffeine, xanthine or |
| 5-MeO-DMT, LSD, DMT, | lives (whichever is longer) before | poppy seeds |
| Syrian Rue or other | study drug administration, except for | Alcohol containing |
| psychedelic agents or | medications that are required for the | products |
| mixtures in their synthetic or | treatment of AEs. Note: Use of | |
| naturally-occurring form | paracetamol (≤3000 mg/day) or | |
| Use of amphetamines, | ibuprofen (≤1200 mg/day) for a | |
| opioids, MDMA | maximum of 3 days is allowed, and | |
| | acute use of topical steroids is also | |
| | allowed. Use of hormonal | |
| | contraceptives for WOCBP is | |
| | allowed. | |
| | Psychoactive medication (e.g., a | |
| | selective serotonin reuptake inhibitor | |
| | such as paroxetine or citalopram), or | |
| | a medication with MAO activity | |
| | (such as isocarboxazid, phenelzine, | |
| | selegiline or tranylcypromine, | |
| | linezolid, and methylene blue) in the | |
| | 28 days prior to study drug | |
| | administration | |
| | Haloperidol, an atypical anti- | |
| | psychotic. | |
| | Immunization/Vaccines (in the 28 | |
| | days prior to study drug | |
| | administration) | |
| | Synthetic or naturally-occurring | |
| | cannabinoids (in the 28 days prior to | |
| | study drug administration) | |
| | Drugs that have the potential to | |
| | precipitate serotonin syndrome, in | |
| | the 28 days prior to study drug | |
| | administration (including the drugs | |
| | listed in Table 5). | |

Abbreviations: 5-MeO-DMT = 5-methoxy-N,N-dimethyltryptamine; AE = Adverse event; DMT = Dimethyltryptamine; EOS = End of study; ET = early termination; LSD = lysergic acid diethylamide (LSD); OTC = over-the-counter; PK = Pharmacokinetics.

TABLE 5
Agents That Alone or in Conjunction With Other Serotonergic
Agents Can Precipitate Serotonin Syndrome

| Drug Class | List of Agents |
|---|---|
| Selective | Citalopram |
| Serotonin | Escitalopram |
| Reuptake | Fluoxetine |
| Inhibitors | Fluvoxamine |
| (SSRIs) | Olanzapine/Fluoxetine (combination product) |
| | Paroxetine |
| | Sertaline |
| Serotonin | Duloxetine |
| Norepinephrine | Desvenlafaxine |
| Reuptake | Sibutramine |
| Inhibitors | Venlafaxine |
| (SNRIs) | Milnacipran |
| | Levomilnacipran, |
| Triptans | Almotriptan |
| | Eletriptan |
| | Frovatriptan |
| | Naratriptan |
| | Rizatriptan |
| | Sumatriptan |
| | Zolmitriptan |
| Miscellaneous | Amphetamines (including: dextroamphetamine, |
| | methamphetamine) |
| | Amphetamine derivatives (including: fenfluramine, |
| | dexfenfluramine, phentermine) |
| | Mirtazapine |
| | Cocaine |
| | MDMA (ecstasy) |
| | Meperidine |
| | Tramadol |
| | Pentazocine |
| | Dextromethorphan |
| | Sibutramine |
| | Bupropion |
| | Serotonin modulators (nefazodone, trazodone, |
| | vilazodone, and vortioxetine) |
| | Cyclic antidepressants (amitriptyline, amoxapine, |
| | clomipramine, desipramine, doxepin, imipramine, |
| | maprotiline, nortriptyline, protriptyline, trimipramine) |
| | St. John's wort (Hypericum perforatum) |
| | 5-HT3 receptor antagonists (dolasetron, granisetron, |
| | ondansetron, palonosetron) |
| | Cyclobenzaprine |
| | Methylphenidate, dexmethylphenidate |
| | Nonselective MAO inhibitors (isocarboxazid, |
| | linezolid, phenelzine, Syrian rue [Peganum harmala, |
| | harmine], and tranylcypromine) |
| | MAO-A inhibitors (methylene blue, moclobemide) |
| | MAO-B inhibitors (rasagiline, safmamide, and |
| | selegiline) |
| | Buspirone |
| | Triptans (almotriptan, eletriptan, frovatriptan, |
| | naratriptan, rizatriptan, sumatriptan, zolmitriptan) |
| | Ergot derivatives (including |
| | dihydroergotamine, ergotamine, |
| | methylergonovine) |
| | Fentanyl |
| | LSD |
| | Lasmiditan |
| | Lorcaserin |
| | Metaxalone |
| | Lithium |

Source: https://www.uspharmacist.com/article/drug-induced-serotonin-syndrome and https://www.uptodate.com/contents/serotonin-syndrome-serotonin-toxicity#H3.

TABLE 6
Summary of AEs

| | RE104 | | | | | | | | |
|---|---|---|---|---|---|---|---|---|---|
| | 5.5 mg | 11 mg | 22 mg | 33 mg | 38 mg | 44 mg | | Placebo | |
| n (%) | (n = 6) | (n = 6) | (n = 6) | (n = 9) | (n = 6) | (n = 3) | | (n = 12) | |
| Any AE | 2 (33.3) | 2 (33.3) | 2 (33.3) | 8 (88.9) | 5 (83.3) | 3 | (100) | 4 | (33.3) |

| Any SAE | 0 | 0 | 0 | 0 | 0 | 0 | | 0 |
|---|---|---|---|---|---|---|---|---|
| Severe AEs | 0 | 0 | 0 | 0 | 1 (16.7) | 1 | (33.3) | 0 |

| | | | | | | | |
|---|---|---|---|---|---|---|---|
| AEs leading to | 0 | 0 | 0 | 0 | 0 | 0 | 0 |

AEs by preferred term (≥2 participants across all dose levels)

| Nausea | 1 (16.7) | 2 (33.3) | 0 | 3 (33.3) | 2 (33.3) | 2 | (66.7) | 0 |
|---|---|---|---|---|---|---|---|---|
| Sinus tachycardia | 0 | 0 | 0 | 4 (44.4) | 3 (50.0) | 2 | (66.7) | 0 |
| Restlessness | 0 | 0 | 0 | 3 (33.3) | 3 (50.0) | 1 | (33.3) | 0 |
| Headache | 0 | 1 (16.7) | 0 | 1 (11.1) | 4 (66.7) | 0 | | 0 |
| Agitation | 0 | 0 | 0 | 0 | 2 (33.3) | 2 | (66.7) | 0 |
| Diarrhea | 0 | 0 | 1 (16.7) | 0 | 2 (33.3) | 0 | | 0 |
| Hyperhidrosis | 0 | 0 | 1 (16.7) | 0 | 1 (16.7) | 1 | (33.3) | 0 |
| Muscle twitching | 0 | 0 | 0 | 3 (33.3) | 0 | 0 | | 0 |
| Abdominal pain | 0 | 0 | 0 | 2 (22.2) | 0 | 0 | | 0 |
| Distress | 0 | 0 | 0 | 0 | 1 (16.7) | 1 | (33.3) | 0 |

| Fatigue | 0 | 0 | 0 | 2 (22.2) | 0 | 0 | 0 | |
|---|---|---|---|---|---|---|---|---|
| Feeling hot | 0 | 0 | 1 (16.7) | 0 | 0 | 0 | 1 | (8.3) |
| Heart rate increased | 0 | 0 | 1 (16.7) | 0 | 0 | 0 | 1 | (8.3) |
| Musculoskeletal injury | 0 | 0 | 0 | 1 (11.1) | 0 | 0 | 1 | (8.3) |

| Thirst | 0 | 0 | 0 | 0 | 2 (33.3) | 0 | | 0 |
|---|---|---|---|---|---|---|---|---|
| Tremor | 0 | 0 | 0 | 1 (11.1) | 0 | 1 | (33.3) | 0 |
| Vomiting | 1 (16.7) | 0 | 0 | 0 | 0 | 1 | (33.3) | 0 |

SAE, serious adverse event

TABLE 7
Summary of MEQ Responders by Dose

| | | Participants with MEQ total | |
|---|---|---|---|
| Dose | n | score ≥60%, % (n) | |
| Placebo | 12 | 8.3 | (1) |
| RE104 | | | |

| | | | | |
|---|---|---|---|---|
| 5.5 | mg | 6 | 0 | (0) |
| 11 | mg | 6 | 50 | (3) |
| 22 | mg | 6 | 16.7 | (1) |
| 33 | mg | 9 | 66.7 | (6) |
| 38 | mg | 6 | 83.3 | (5) |
| 44 | mg | 3 | 100 | (3) |

MEQ, Mystical Experience Questionnaire.

TABLE 8
Pharmacokinetics Mean Values (Cohorts 1-6)

| Cohort | | | | | |
|---|---|---|---|---|---|
| 1 | 2 | 3 | 4 | 5 | 6 |
| RE104 dose | | | | | |
| 5.5 mg | 11 mg | 22 mg | 33 mg | 38 mg | 44 mg |
| (n = 6) | (n = 6) | (n = 6) | (n = 9) | (n = 6) | (n = 3) |

RE104 (prodrug), mean (CV %)

| | | | | | | | | | | | | |
|---|---|---|---|---|---|---|---|---|---|---|---|---|
| Cₘₐₓ, ng/mL | 173 | (60) | 527 | (39) | 788 | (16) | 1461 | (56) | 1246 | (22) | 1805 | (20) |
| Tₘₐₓ, h | 0.42 | (31) | 0.33 | (39) | 0.38 | (36) | 0.25 | (45) | 0.38 | (36) | 0.42 | (35) |
| t_{1/2}, h | 0.53 | (28) | 0.48 | (21) | 0.50 | (24) | 0.43 | (14) | 0.64 | (40) | 0.52 | (13) |

4-OH-DiPT (active drug), mean (CV %)

| | | | | | | | | | | | | |
|---|---|---|---|---|---|---|---|---|---|---|---|---|
| Cₘₐₓ, ng/mL | 18 | (29) | 60 | (35) | 77 | (42) | 137 | (38) | 124 | (28) | 275 | (9) |
| Tₘₐₓ, h | 1.12 | (23) | 1.21 | (40) | 1.42 | (58) | 1.07 | (42) | 1.08 | (48) | 1.25 | (20) |
| t_{1/2}, h | 3.42 | (63) | 3.55 | (51) | 3.27 | (83) | 2.79 | (49) | 2.79 | (49) | 3.76 | (54) |

Cₘₐₓ, maximum plasma concentration; CV, coefficient of variation; t_{1/2}, half-life; Tₘₐₓ, time to maximum plasma concentration.

### Supplemental Tables

SUPPLEMENTAL TABLE 1
Summary of Mean Pharmacokinetic Parameters Estimated for
4-OH-DiPT After 2 mg/kg RE104 Administration in Rats

| Parameter, mean (SD) | IV administration | | PO administration | | SC administration | |
|---|---|---|---|---|---|---|
| tₘₐₓ, h | 0.0833 | (0.00) | 1.00 | (0.00) | 1.00 | (0.00) |
| Cₘₐₓ, ng/ml | 305 | (28.6) | 0.304 | (0.100) | 150 | (51.2) |
| Apparent t_{1/2}, h | 0.601 | (0.0214) | NA | | 0.674 | (0.0373) |
| AUC₀₋ₗₐₛₜ, h · ng/mL | 146 | (4.62) | 0.242 | (0.122) | 203 | (42.8) |

| | | | | | |
|---|---|---|---|---|---|
| AUC₀₋₋, h · ng/mL | 147 | (4.50) | NA | 204 | (42.9) |
| MRT₀₋₋, h | 0.695 | (0.0206) | NA | 1.33 | (0.0166) |

| | | | | |
|---|---|---|---|---|
| fₘ, %ₐ | 96.7 | (2.96) | NA | NA |

4-OH-DiPT, 4-hydroxy-N,N-diisopropyltryptamine; AUC₀₋₋, area under the curve from the time of dosing extrapolated to infinity; AUC₀₋ₗₐₛₜ, area under the curve from the time of dosing to tlast; Cₘₐₓ, maximum observed concentration after nonparenteral administration; fₘ, fraction metabolized (AUC^{iv}[parent]ₚ_{^{rodru}}_{g}/AUC ^{iv}[parent]ₚ_{^{arent}}, at equimolar doses); IV, intravenous; MRT₀₋₋, mean residence time from the time of dosing extrapolated to infinity; PO, per os (oral); NA, not applicable; SC, subcutaneous; SD, standard deviation; t_{1/2}, half-life; tₘₐₓ, time of maximum observed concentration after nonparenteral administration.
ₐCalculated from AUC₀₋₋(IV) for 4-OH-DiPT (152 h · ng/mL) administered at an equimolar dose.

SUPPLEMENTAL TABLE 2
Summary of Mean Pharmacokinetic Parameters Estimated for 4-
OH-DiPT After 1.39 mg/kg 4-OH-DiPT Administration in Rats

| Parameter, mean (SD) | IV administration | | PO administration | | SC administration | |
|---|---|---|---|---|---|---|
| C₀, ng/ml | 458 | (54.9) | NA | | NA | |
| tₘₐₓ, h | NA | | 0.250 | (0.00) | 0.611 | (0.474) |
| Cₘₐₓ, ng/mL | NA | | 1.88 | (0.868) | 103 | (21.6) |
| Apparent t_{1/2}, h | 0.740 | (0.159) | 0.478 | (0.0891) | 0.674 | (0.104) |
| AUC₀₋ₗₐₛₜ, h · ng/mL | 152 | (33.1) | 1.07 | (0.710) | 134 | (64.3) |
| AUC₀₋₋, h · ng/mL | 152 | (33.1) | 1.26 | (0.700) | 135 | (64.8) |
| CL, mL/h/kg | 9440 | (2240) | NA | | NA | |
| MRT₀₋₋, h | 0.6440 | (0.118) | 0.746 | (0.0775) | 1.18 | (0.525) |

| | | | | |
|---|---|---|---|---|
| Vₛₛ, mL/kg | 5910 | (387) | NA | NA |

| | | | | | |
|---|---|---|---|---|---|
| F, % | 100 | 0.825 | (0.459) | 88.6 | (42.5) |

4-OH-DiPT, 4-hydroxy-N,N-diisopropyltryptamine; AUC₀₋₋, area under the curve from the time of dosing extrapolated to infinity; AUC₀₋ₗₐₛₜ, area under the curve from the time of dosing to tlast; C₀, concentration extrapolated to time zero after IV dose; CL, clearance; Cₘₐₓ, maximum observed concentration after nonparenteral administration; F, oral bioavailability ([Dose^{IV} × AUC ^{PO}[/[Dose^{PO} × AUC^{IV}[×100); IV, intravenous; MRT₀₋₋, mean residence time from the time of dosing extrapolated to infinity; PO, per os (oral); NA, not applicable; SC, subcutaneous; SD, standard deviation; t_{1/2}, half-life; tₘₐₓ, time of maximum observed concentration after nonparenteral administration, Vₛₛ, volume of distribution at steady state.

SUPPLEMENTAL TABLE 3
Summary of Plasma Pharmacokinetic Parameters of RE104
and 4-OH-DiPT In Dogs After RE104 Administration

| RE104 dose | | | | | Tₗₐₛₜ, h, | | | AUC_{0-72 h}, | | AUC_{0-∞}, | |
|---|---|---|---|---|---|---|---|---|---|---|---|
| and route of | tₘₐₓ, h, median | | Cₘₐₓ, ng/mL, | | median | AUC₀₋ₜ, | | h · ng/mL, | | h · ng/mL, | |
| administration | (range) | | mean (SD) | | (range) | h · ng/mL | | mean (SD) | | mean (SD) | |
| 1 mg/kg IV | | | | | | | | | | | |
| RE104 | 0.083 | (0.083, 0.083) | 1870 | (722) | 2 (1.2) | 562 | (222) | 572 | (217) | 568 | (218) |
| 4-OH-DiPT | 0.083 | (0.083, 0.482) | 86.2 | (20.6) | 4 (4.8) | 134 | (60.7) | 137 | (62.0) | 135 | (61.3) |
| 1 mg/kg SC | | | | | | | | | | | |
| RE104 | 0.500 | (0.250, 0.500) | 344 | (160) | 8 (4.8) | 621 | (33.7) | 658 | (70.9) | 645 | (64.5) |
| 4-OH-DiPT | 2.000 | (0.500, 2.000) | 34.0 | (7.71) | 8 (4.8) | 132 | (53.4) | 155 | (78.6) | 95.2 | (NC)^{a} |
| 5 mg/kg PO | | | | | | | | | | | |
| RE104 | 0.500 | (0.250, 2.00) | 12.2 | (6.11) | 4 (2.4) | 20.0 | (5.56) | 24.1 | (6.66) | 21.8 | (NC)^{b} |

| | | | | | | | | | | |
|---|---|---|---|---|---|---|---|---|---|---|
| 4-OH-DiPT | 4.000 | (2.000, 4.000) | 21.4 | (11.5) | 8 (4.8) | 103 | (69.1) | 120 | (76.8) | NC^{c} |

| RE104 dose | | | | V_{d}, L, | F, % | | MR, |
|---|---|---|---|---|---|---|---|
| and route of | t_{1/2}, h, mean | | CL, L/h, | mean | mean | | mean |
| administration | (SD) | | mean (SD) | (SD) | (SD) | | (SD) |
| 1 mg/kg IV | | | | | | | |
| RE104 | 0.255 | (0.00 [symbol] 36) | 23.4 (0.627) | 8.61 (0.106) | NA | | — |
| 4-OH-DiPT | 0.863 | (0.401) | — | — | — | | 0.339 (0.223) |
| 1 mg/kg SC | | | | | | | |
| RE104 | 1.30 | (0.816) | NA | NA | 121 | (27.2) | — |
| 4-OH-DiPT | 0.888 | (NC)^{a} | — | — | — | | 0.332 (0.131) |
| 5 mg/kg PO | | | | | | | |
| RE104 | 1.30 | (NC)^{b} | NA | NA | 0.854 | (0.214) | — |

| | | | | | |
|---|---|---|---|---|---|
| 4-OH-DiPT | NC^{c} | — | — | — | 7.04 (3.18) |

AUC₀₋ₜ, area under the concentration-time curve from time 0 to last time point; AUC_{0-72 h}, area under the concentration-time curve from time 0 to 72 hours; AUC_{0-∞}, area under the concentration-time curve from time 0 to infinity; CL, clearance; Cₘₐₓ, maximum plasma exposure; F, bioavailability; IV, Intravenous; MR, metabolic ratio; NA, not applicable; NC, not calculated; SC, subcutaneous; SD, standard deviation, t_{1/2}, elimination half-life; tₗₐₛₜ, time last measured; tₘₐₓ, time of maximum exposure; V_{d}, volume of distribution.
^{a}n = 1; AUC_{0-∞} and t_{1/2} could not be reliably determined for 2 males because there were fewer than 3 measurable concentrations after Cₘₐₓ.
^{b}n = 2; AUC_{0-∞} and t_{1/2} could not be reliably determined for 1 male because there were fewer than 3 measurable concentrations after Cₘₐₓ.
^{c}n = 0; AUC_{0-∞} and t_{1/2} could not be reliably determined for any males because there were fewer than 3 measurable concentrations after Cₘₐₓ. [symbol]
indicates data missing or illegible when filed

What is claimed is:

1. An injectable composition comprising a compound of Formula (I): 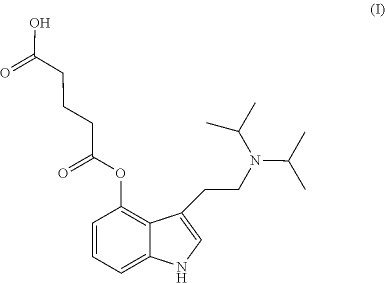 [SMILES: CC(C)N(CCc1c[nH]c2cccc(OC(=O)CCCC(=O)O)c12)C(C)C]
or a pharmaceutically acceptable salt thereof, wherein the compound of Formula (I) is present at a dose of from about 30 mg to about 50 mg calculated as the free base, together with a pharmaceutically acceptable carrier.

2. An injectable composition comprising a compound of Formula (I): 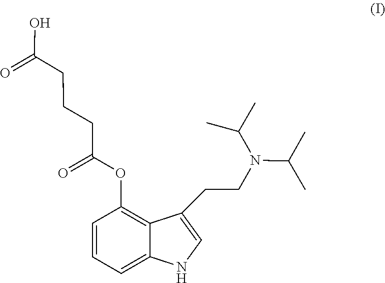 [SMILES: CC(C)N(CCc1c[nH]c2cccc(OC(=O)CCCC(=O)O)c12)C(C)C]
or a pharmaceutically acceptable salt thereof, wherein the compound of Formula (I) is present at a dose of 30 mg or 40 mg calculated as the free base, together with a pharmaceutically acceptable carrier.

3. The injectable composition defined in claim 1 or claim 2, wherein the compound of Formula (I) is the hydrochloride salt thereof.

4. The injectable composition defined in claim 2, wherein the compound of Formula (I) is the hydrochloride salt thereof present at a dose of 33 mg or 44 mg.

5. The injectible composition defined in any one of claims 1-4, wherein wherein the injectable composition is a subcutaneous injectable composition.

6. A method of treating a mental disorder comprising the step of administering to a patient in need of treatment a pharmaceutical composition comprising a compound of Formula (I): 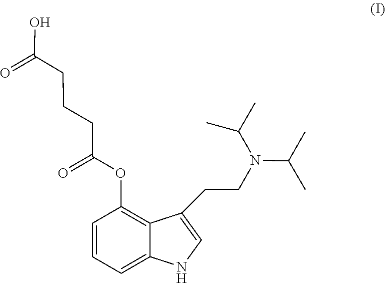 [SMILES: CC(C)N(CCc1c[nH]c2cccc(OC(=O)CCCC(=O)O)c12)C(C)C]
at a dose of from about 30 mg to about 50 mg calculated as the free base, together with a pharmaceutically acceptable carrier.

7. A method of treating a mental disorder comprising the step of administering to a patient in need of treatment a pharmaceutical composition comprising a compound of Formula (I): 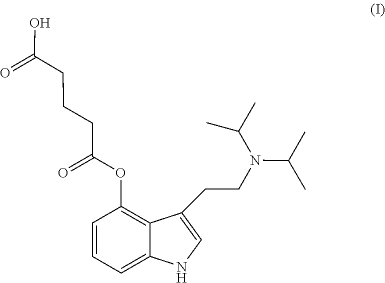 [SMILES: CC(C)N(CCc1c[nH]c2cccc(OC(=O)CCCC(=O)O)c12)C(C)C]
at a dose of about 30 mg or about 40 mg calculated as the free base, together with a pharmaceutically acceptable carrier.

8. The method defined in claim 6 or claim 7, wherein the compound of Formula (I) is the hydrochloride salt thereof.

9. The method defined in claim 7, wherein the compound of Formula (I) is the hydrochloride salt thereof present at a dose of 33 mg or 44 mg.

10. A method of treating a mental disorder comprising the step of administering to a patient in need of treatment a pharmaceutical composition comprising a compound of Formula (I): 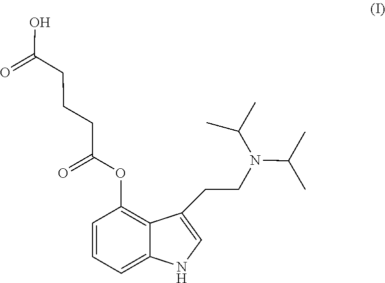 [SMILES: CC(C)N(CCc1c[nH]c2cccc(OC(=O)CCCC(=O)O)c12)C(C)C]
at a dose of 1 mg/kg calculated as the free base, together with a pharmaceutically acceptable carrier.

11. The method defined in any one of claims 6-10, wherein the compound of Formula (I) has a duration of action of from about 2.5 hours to about 4.5 hours after administration.

12. The method defined in any one of claims 6-10, wherein the compound of Formula (I) has a duration of action of about 3.5 hours or 3.7 hours after administration.

13. The method defined in any one of claims 6-12, wherein the compound of Formula (I) has a Cₘₐₓ in the patient in the range of from about 1000 ng/mL to about 2000 ng/mL.

14. The method defined in any one of claims 6-12, wherein the compound of Formula (I) has a Cₘₐₓ in the patient in the range of from about 1500 ng/mL to about 1800 ng/mL.

15. The method defined in any one of claims 6-14, wherein the compound of Formula (I) has a Tₘₐₓ in the patient in the range of from about 20 minutes 30 minutes.

16. The method defined in any one of claims 6-14, wherein the compound of Formula (I) has a Tₘₐₓ in the patient in the range of about 15 minutes.

17. The method defined in any one of claims 6-16, wherein the compound of Formula (I) is converted to a compound of Formula (II) after administration 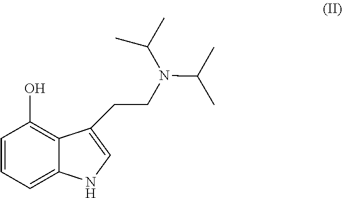 [SMILES: CC(C)N(CCc1c[nH]c2cccc(O)c12)C(C)C]
and the compound of Formula (II) has a Cₘₐₓ in the patient in the range of from about 100 ng/mL to about 300 ng/mL.

18. The method defined in any one of claims 6-16, wherein the compound of Formula (I) is converted to a compound of Formula (II) after administration 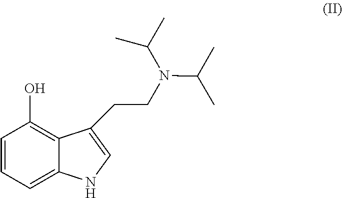 [SMILES: CC(C)N(CCc1c[nH]c2cccc(O)c12)C(C)C]
and the compound of Formula (II) has a Cₘₐₓ in the patient in the range of from about 120 ng/mL to about 300 ng/mL.

19. The method defined in any one of claims 6-18, wherein the compound of Formula (I) is converted to a compound of Formula (II) after administration 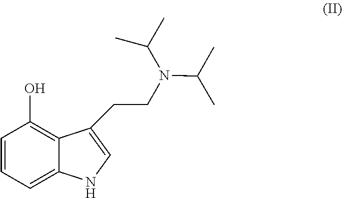 [SMILES: CC(C)N(CCc1c[nH]c2cccc(O)c12)C(C)C]
and the compound of Formula (II) has a Tₘₐₓ in the patient in the range of from about 60 minutes to about 150 min.

20. The method defined in any one of claims 6-18, wherein the compound of Formula (I) is converted to a compound of Formula (II) after administration 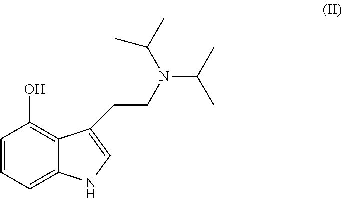 [SMILES: CC(C)N(CCc1c[nH]c2cccc(O)c12)C(C)C]
and the compound of Formula (II) has a Tₘₐₓ in the patient in the range of from about 60 minutes to about 80 min.

21. The method defined in any one of claims 6-20, which demonstrates an average MEQ30 score in the patient of at least 50%.

22. The method defined in any one of claims 6-20, which demonstrates an average MEQ30 score in the patient of at least 60%.

23. The method defined in any one of claims 6-22, wherein the pharmaceutical composition is an injectable pharmaceutical composition.

24. The method defined in any one of claims 6-22, wherein the pharmaceutical composition is an subcutaneous injectable pharmaceutical composition.
